(19) United States
(12) Patent Application Publication
(10) Pub. No.: US 2017/0181905 A1
(43) Pub. Date: Jun. 29, 2017

(54) FASTENING SYSTEM

(71) Applicant: 3M INNOVATIVE PROPERTIES COMPANY, St. Paul, MN (US)

(72) Inventors: Hiroshi Sakurai, Kanagawa (JP); Yoshihisa Matsuda, Kanagawa (JP); Masato Kondo, Kanagawa (JP); Hirofumi Hosokawa, Tokyo (JP); Yoshiyuki Matsumura, Kanagawa (JP); Shinji Kimura, Kanagawa (JP)

(21) Appl. No.: 15/304,955

(22) PCT Filed: Apr. 10, 2015
(86) PCT No.: PCT/US2015/025297

§ 371 Date: Oct. 18, 2016

(30) Foreign Application Priority Data
Apr. 21, 2014 (JP) 2014-087361

(51) Int. Cl. A61F 13/62; A61F 13/56; A44B 18/00

(52) U.S. Cl. CPC A61F 13/622; A44B 18/0046; A44B 18/0065; A44B 18/0057; A61F 13/5622

(57) ABSTRACT

A fastening system for securing a first region having a first securing member and a second region having a second securing member. The system includes a first surface fastener provided on a first securing member and a second surface fastener provided on a first securing member or a second securing member. That securing member, out of the first securing member and the second securing member, having the second surface fastener provided thereon includes the second surface fastener and a substrate having the second surface fastener laminated thereon.

### FIELD

One aspect of the present invention relates to a fastening system provided with a first surface fastener and a second surface fastener.

### BACKGROUND

Conventionally, surface fasteners have been used for securing different regions in a member to be secured such as fabric and for securing a plurality of members to be secured and so on.

U.S. Pat. No. 4,699,622 listed below discloses a disposable diaper having an improved side closure. U.S. Pat. No. 4,699,622 describes, “The disposable diaper of the present invention is provided with an outer fastening means and with an inner fastening means”.

U.S. Pat. No. 6,613,032 listed below discloses an absorbent product or a product capable of holding an absorbent material. U.S. Pat. No. 6,613,032 describes, “A product according to the present invention allows the front securing members and the back securing members to be located such that the front securing members and the back securing members only partially receive each other when the product is fastened”.

U.S. Pat. No. 6,654,994 discloses indicia for a garment with a dual fastening system. U.S. Pat. No. 6,654,994 describes, “The dual fastening system includes at least two first fasteners and at least two second fasteners, at least a portion of each second fastener being located in the front waist region and situated inboard from each longitudinal side edge of the front waist region, each second fastener being configured to engage at least a portion of the bodyfacing surface”.

U.S. Pat. No. 7,662,137 listed below discloses a disposable garment provided with multiple fasteners. U.S. Pat. No. 7,662,137 also describes, “The dual fastening system includes at least two first fasteners and at least two second fasteners, at least a portion of each second fastener being located in the front waist region and situated inboard from each longitudinal side edge of the front waist region, each second fastener being configured to engage at least a portion of the bodyfacing surface.”

U.S. Pat. No. 7,569,042 listed below discloses a disposable diaper. U.S. Pat. No. 7,569,042 describes, “In one of front and rear waist regions of a disposable diaper, first wings which are elastically stretchable in a waist-surround direction are formed and a first fastener means is provided on the body facing surfaces of the first wings. The other waist region is provided on its undergarment facing surface with, in addition to a second fastener means, anti-slip zones exhibiting desired average kinetic friction force relative to the body facing surfaces of the first wings.”

US Published Application 2013/0310794 listed below discloses a wear absorbent article with tabs. US Published Application 2013/0310794 describes, “A primary fastening system is provided for securing the article in a wear configuration having a waist opening and a pair of leg openings spaced from the waist opening. A secondary fastening system comprises a pair of tabs having a fastener region selectively attachable to the article and an attachment region defining a portion of the primary fastening system.”

### SUMMARY

The above-mentioned patent documents disclose using two surface fasteners at a securing point to prevent fastener tabs from folding or the fasteners coming off due to a wearer's movement or the like (i.e., firmly securing a garment or absorbent article to the wearer). However, using two fasteners at a single securing point may degrade the securement of a member to be secured and the conformity of linking portions with respect to movement of the member to be secured. Moreover, as a result, phenomena such as a portion of the surface fastener engaged with the member to be secured separating from the member to be secured can more easily occur. If the member to be secured is an absorbent article or a garment, the surface fastener will be harder than the absorbent article or garment and may irritate the skin when the article or garment is used or worn.

It is therefore desirable for a securing part to more reliably conform to movement of a member to be secured while maintaining stability against shifting on the securing part.

A fastening system according to one aspect of the present invention is for securing a first region having a first securing member and a second region having a second securing member. The fastening system is provided with a first surface fastener provided on the first securing member and a second surface fastener provided on the first securing member or the second securing member. That securing member, out of the first securing member and the second securing member, having the second surface fastener provided thereon includes the second surface fastener and a substrate having the second surface fastener laminated thereon. Defining Sa as bending stiffness of a laminated section formed by the second surface fastener and the substrate and Sb as bending stiffness of a comparison member constituted by a laminate formed from a substrate having no second surface fastener laminated thereon and a piece of cellophane adhesive tape as specified in JIS Z 1522, the relationship Sa/Sb<2.0 holds.

According to such an aspect, the laminated section formed by the substrate of the securing member upon which the second surface fastener is provided and the surface fastener has low bending stiffness, with the result that, when the two regions are secured, the hold yielded by the fastening system flexibly deforms in response to bending or twisting generated by movement of the member to be secured. It is thus possible to ensure reliable conformity of the securing part with respect to movement of a member to be secured even when two surface fasteners are used to increase stability against shifting. Irritation of the skin of the wearer can also be minimized in cases in which the fastening system is used with an absorbent article or a garment.

In accordance with an aspect of the present invention, it is possible for a securing part to more reliably conform to movement of a member to be secured while maintaining stability against shifting on the securing part. Irritation of the skin of the wearer can also be minimized in cases in which the fastening system is used with an absorbent article or a garment.

### BRIEF DESCRIPTION OF DRAWINGS

FIG. 1 is a perspective view of a diaper provided with a fastening system according to an embodiment.

FIG. 2 is a perspective view illustrating a state in which the diaper of FIG. 1 is being worn.

FIG. 3 is a side view of an example of a hook.

FIG. 4 is an illustration of an example of a hook member.

FIG. 5 is an illustration of another example of a hook member.

FIGS. 6A to 6C are cross-sectional views along line VI-VI in FIG. 5.

FIG. 7 is an illustration of still another example of a hook member.

FIG. 8 is an illustration of yet another example of a hook member.

FIG. 9 is an illustration of yet another example of a hook member.

FIG. 10 is an illustration of yet another example of a hook member.

FIG. 11 is an illustration of yet another example of a hook member.

FIG. 12 is an illustration of yet another example of a hook member.

FIGS. 13A to 13C are cross-sectional views along line XIII-XIII in FIG. 12.

FIG. 14 is an illustration of yet another example of a hook member.

FIG. 15 is an illustration of yet another example of a hook member.

FIG. 16 is an illustration of yet another example of a hook member.

FIG. 17 is an illustration of yet another example of a hook member.

FIG. 18 is an illustration of yet another example of a hook member.

FIG. 19 is an illustration of a hook member in an elongated state.

FIG. 20 is an illustration of yet another example of a hook member.

FIG. 21 is an illustration of an example of a method of measuring the bending stiffness of a securing member.

FIG. 22 is an illustration of an example of a method of measuring the bending stiffness of a securing member.

FIG. 23 is an illustration of an example of a securing element using a hook member.

FIG. 24 is a perspective view of a diaper provided with a fastening system according to a modified example.

FIG. 25 is an illustration of diapers provided with a fastening system according to a modified example.

FIG. 26 is an illustration of diapers provided with a fastening system according to a modified example.

### DETAILED DESCRIPTION

Embodiments of the present invention will now be described in detail with reference to the attached drawings. In the descriptions of the drawings, identical or similar parts are assigned the same reference number, and redundant descriptions thereof will be omitted.

In the context of the present specification, the term “securing member” refers to a member used to overlap and secure first and second regions separated from each other. For example, as used in the present specification, the term “securing member” is used in applications such as: (1) securing first and second regions of a single article (specific examples including joining front and rear waist sections of an absorbent article, such as a disposable diaper, securing left and right front body sections, collars and/or sleeve cuffs of a garment, and securing a foot opening in the upper of a shoe), (2) securing a first region present on one article and a second region present on another article (specific examples including securing or attaching an absorbent article, such as a sanitary article and other hygienic article, to a garment), (3) securing (hold in place) a member to be secured by wrapping a securing member around the member to be secured or by engaging a surface fastener with a substrate (specific examples including maintaining the shape of an article that has been folded or rolled into a small package and preventing shifting or peeling of a patch applied to the skin), and the like. However, the range encompassed by the term “securing member” is not limited to these examples.

The first region includes a first securing member, and the second region includes a second securing member. The first securing member includes at least a substrate and a first surface fastener provided upon the substrate. Meanwhile, the second securing member includes at least a substrate. A second surface fastener is provided upon the substrate of the first securing member or the substrate of the second securing member. As such, conceivable embodiments of the fastening system include one in which the first securing member is provided with both the first surface fastener and the second surface fastener, and no surface fastener is provided on the second securing member, and one in which the first securing member is provided with the first surface fastener, and the second securing member is provided with the second surface fastener. In other words, the term “securing member” is used in the present specification even when the member is provided with neither the first surface fastener nor the second surface fastener. As described above, the first region and the second region includes the first securing member and the second securing member, respectively, and are secured by a fastening system provided with the first surface fastener and the second surface fastener provided, as described above, on these securing members. In the present specification, the term “substrate” refers to a base (foundation) member for a securing member to which a surface fastener is or can be attached. The parts of the securing member in the through-thickness direction other than the surface fastener can also be referred to as the “substrate”.

The fastening system according to the present embodiment is provided with at least two surface fasteners. One of the at least two surface fasteners is flexible. The fastening system according to the present embodiment allows a securing part to more reliably conform to the movement of a member to be secured while maintaining stability against shifting on the securing part. In the context of the present specification, the term “securing part” refers to a part where the first region and the second region are attached to each other (i.e., secured) by the at least two surface fasteners.

In the present embodiment, the flexibility of the surface fastener is represented by the bending stiffness (Sa) of the laminated section formed by the substrate and the surface fastener laminated to the substrate of the securing member provided with the surface fastener. The bending stiffness Sa is indicated by using a laminate constituted by the substrate used in the laminated section to which a piece of cellophane adhesive tape as specified in JIS Z 1522 (hereafter, “comparison tape”) has been laminated, taking the bending stiffness Sb thereof as a baseline, and determining the ratio Sa/Sb. The second surface fastener of the present specification is the most flexible of the surface fasteners with which the fastening system is provided. More specifically, the second surface fastener has the lowest Sa/Sb.

It will be readily apparent to a person skilled in the art that it is not necessarily required that Sa/Sb for all of the surface fasteners of the fastening system be determined in order to identify the second surface fastener of the fastening system. For example, visual observation may be used to determine which surface fastener is the second surface fastener, or the locations where the various surface fasteners are provided may be touched or bent by hand to determine which surface fastener is the second surface fastener. Alternatively, the second surface fastener can be identified by measuring the bending stiffness of the surface fastener itself. Note that bending stiffness may also be determined according to loop stiffness or Gurley stiffness as described hereafter, or according to another standard. It goes without saying that if it would be difficult to make the determination using the methods described above, the second surface fastener may be identified by determining the above Sa/Sb as a last resort.

Note that if there are a plurality of surface fasteners having the smallest Sa/Sb in the fastening system, any surface fastener selected from among the plurality of surface fasteners may be designated the second surface fastener.

The relationship Sa/Sb<2.0 holds for the second surface fastener. In other words, defining Sa as the bending stiffness of a laminated section formed by the second surface fastener and the substrate and Sb as the bending stiffness of a comparison member constituted by a laminate formed from a substrate having no second surface fastener laminated thereon and a piece of cellophane adhesive tape as specified in JIS Z 1522, the relationship Sa/Sb<2.0 holds.

The bending stiffnesses Sa, Sb and the ratio Sa/Sb will be discussed in greater detail below.

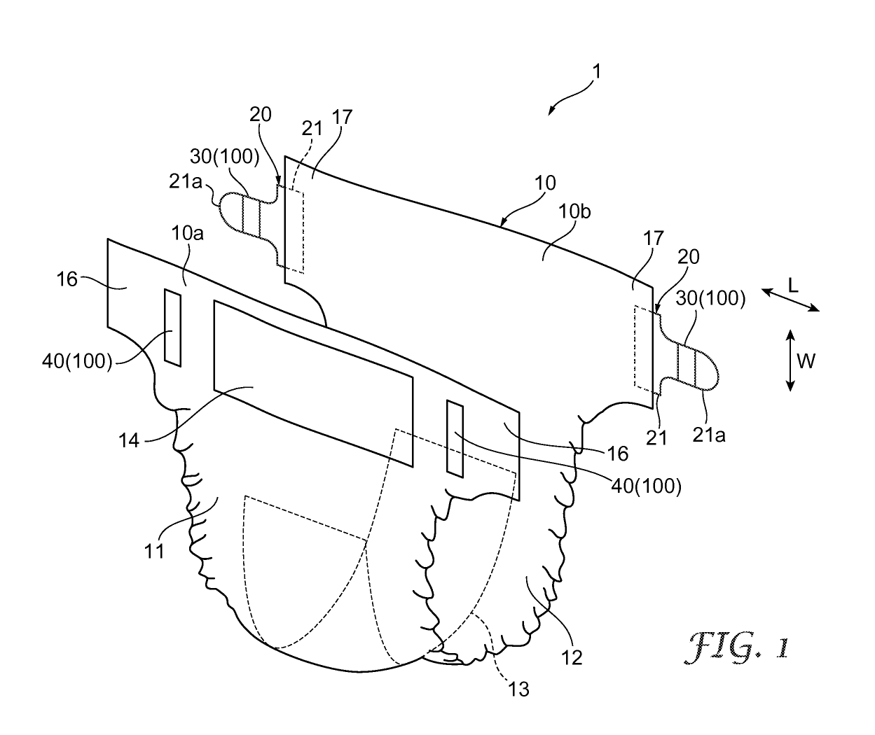

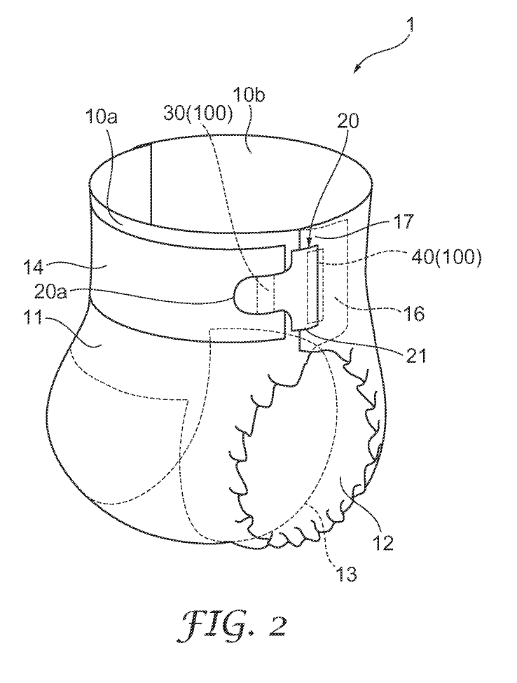

In the present embodiment, an absorbent article 1 illustrated in FIGS. 1 and 2 will be described. FIGS. 1 and 2 illustrate a diaper of open (flat) type as an example of the absorbent article 1, but an absorbent article provided with securing members is not limited to the diaper illustrated in FIGS. 1 and 2. For example, the absorbent article may be a diaper of the other type, such as a tape-fastening type and a pad type, or a sanitary article/hygienic article, such as a sanitary napkin and a shorts-integrated napkin.

The diaper (absorbent article) 1 is provided with a sheet-shaped body section 10 that covers the crotch of the wearer from belly to back. In the present specification, the surface of the diaper 1 facing the wearer will be referred to as the “inner surface”, and the surface on the opposite side as the “outer surface”.

The body section 10 has an outer sheet 11, a liquid-permeable inner sheet 12 laminated to the inner side of the outer sheet 11, and a liquid-absorbent high polymer absorbent body 13 housed between the outer sheet 11 and the inner sheet 12. The outer sheet 11 is typically constituted by a laminate formed from a liquid-impermeable film and a nonwoven fabric layer, the liquid-impermeable film being present on the side where the high polymer absorbent body 13 is present, and the nonwoven fabric layer being present on the outer surface of the body of the diaper 1. In other words, when the diaper 1 is worn, the inner sheet 12 is disposed on the inner surface side of the body of the diaper 1 adjacent to the skin of the wearer, while the outer sheet 11 (the nonwoven fabric layer thereof) is disposed on the outer surface of the body of the diaper 1 adjacent to the clothing or the like of the wearer. For example, a polypropylene nonwoven fabric produced using a spunbond method can be used as the nonwoven fabric layer used in the outer sheet 11. In such cases, the nonwoven fabric layer can be used for securing with the securing member.

A ventral end 10a includes a ventral side panel 16 that extends along a widthwise direction (the lateral direction when the diaper is being worn) of the body section 10. Moreover, a dorsal end 10b includes a dorsal side panel 17 that extends along the widthwise direction of the body section 10. As illustrated in several of the drawings of the present specification, a direction in which the two types of side panels 16, 17 are wrapped is labeled L, and a direction orthogonal to the wrapping direction L is defined as a widthwise direction W. The wrapping direction L is identical to the direction in which the side panels 16, 17 project.

A strip-shaped loop member 14 extending in the widthwise direction of the body section 10 may be provided on the outer surface of the ventral end 10a of the body section 10. In one embodiment, the loop member 14 has a plurality of loop bodies with which hooks engage. Alternatively, a loop member 14 of nonwoven fabric or the like having fibers with which such hooks can engage may be used instead of the loop member 14 having a plurality of loop bodies with which the hooks engage. Moreover, instead of providing a loop member 14, an outer surface layer of the outer sheet 11 may also be constituted by a material provided with a multiplicity of loop bodies with which the hooks can engage, or, as discussed above, the outer sheet 11 may be constituted by a nonwoven fabric or the like having fibers with which the hooks can engage.

A pair of securing members 20 that releasably join the ventral end 10a and the dorsal end 10b of the body section 10 at the left and right sides of the waist of the wearer are provided, by being glued, sewn, melt-bonded or the like, at ends of the dorsal side panel 17 in the wrapping direction L. In the present embodiment, the securing members 20 are also referred to as “fastening tabs”. The securing member 20 is provided with a substrate 21 and a first surface fastener 30 provided on the substrate 21. Meanwhile, second surface fasteners 40 are provided on the outer surface of the ventral side panel 16. Thus, in the diaper 1, the securing members 20 are the first securing members, the dorsal end 10b and the dorsal side panel 17 extending from the dorsal end 10b in the widthwise direction of the body section 10 constitute the first region, the ventral side panel 16 is the second securing member, and the ventral end 10a is the second region. The first surface fasteners 30 engage with the loop member 14 or the ventral end 10a, and the second surface fasteners 40 engage with the dorsal side panel 17. Accordingly, the fastening system 100 according to the present embodiment is for securing the dorsal end 10b having the dorsal side panel 17 and the ventral end 10a having the ventral side panel 16, the system being provided with the first surface fasteners 30 provided on the securing members 20 projecting from the dorsal side panel 17 and the second surface fasteners 40 provided on the ventral side panel 16. When the securing members 20 are wrapped around the loop member 14, the second surface fasteners 40 engage with the dorsal side panel 17, and the first surface fasteners 30 engage with the loop member 14. Thus, when the two regions have been secured by the fastening system 100, the second surface fasteners 40 are positioned farther from free ends 20a of the securing members 20 on which the first surface fasteners 30 are provided than the first surface fasteners 30 (see FIG. 2). In this instance, the free ends 20a of the securing members 20 are the ends of the securing members 20 with respect to the wrapping direction L, and are the ends on the sides of the members not attached to the dorsal side panel 17. The first surface fasteners 30 and the second surface fasteners 40 are both surface fasteners. Surface fasteners can be attached and released within a desired surface range. The concept of “surface fastener” encompasses both hook members and loop members. Note that, in the fastening system 100, the first surface fasteners 30 and the second surface fasteners 40 are not intended to engage with each other. The following description of the present embodiment assumes that the first surface fasteners 30 and the second surface fasteners 40 are both hook members.

The substrate 21 protrudes outward from the dorsal end 10b in the widthwise direction of the body section 10, and wraps around and joins to the ventral end 10a when the diaper is being worn. The substrate 21 is flexible. Here, flexibility refers to a property that allows the substrate 21 to easily bend and conform to the shape of the surface of the region to which the securing member is applied. The substrate 21 may be extendable or stretchable, or may elastically deform. Note that such elastic deformation may include partial plastic deformation. In cases in which the securing members 20 are applied to an absorbent article or a garment, flexibility refers to a level of stiffness that the wearer does not experience an uncomfortably stiff or bulky feel. Note that the substrate 21 may be imparted with a desired degree of flexibility according to the surface shape, structure, composition, use, and the like, of the region to which the securing member is applied. If a nonwoven fabric is used as the substrate 21, flexibility can be expressed in terms of the basis weight of the nonwoven fabric. In some embodiments, the substrate 21 can be viewed as being flexible if the basis weight of the nonwoven fabric is in a range of 5 g/m² or greater, 10 g/m² or greater, or 20 g/m² or greater and 600 g/m² or less, 400 g/m² or less, or 200 g/m² or less. In some embodiments, the nonwoven fabric preferably has a basis weight of 30 g/m² or more and 100 g/m² or less.

In the present embodiment, the substrate 21 has a shape that tapers in the direction of protrusion (wrapping direction), but the substrate 21 is not limited to such a shape. For example, the substrate 21 may be rectangular in shape, or at least part of the outer edges of the substrate 21 may be arc-shaped.

An appropriate material may be selected for the substrate 21 for the sake of the flexibility of the substrate 21 with respect to the region to which the securing member is applied. For example, the substrate 21 may be made of a woven fabric, a nonwoven fabric, a plastic film, or a mixture thereof. The substrate 21 may be made of the same material as the outer sheet 11 and the dorsal side panel 17, in which case the substrate 21, the outer sheet 11, and the dorsal side panel 17 may be formed as a single piece. Alternatively, the substrate 21 having stretchability may be made of a laminate of an elastomeric molded body and fiber aggregate having stretchability. The elastomeric molded body and the fiber aggregate may be joined by a bonding agent, thermal fusion bonding, or the like, or may be joined through a physical method such as weaving, sewing, or the like.

An example of an elastomer is a thermoplastic elastomer. The thermoplastic elastomer consists of a hard segment and a soft segment, and the hard segment primarily has the function of molecular constraint. The thermoplastic elastomer can be classified according to the type of the hard segment thereof. Examples of thermoplastic elastomers include styrene-based thermoplastic elastomers, olefin-based thermoplastic elastomers (TPO), vinyl chloride-based thermoplastic elastomers, urethane-based thermoplastic elastomers, ester-based thermoplastic elastomers, amide-based thermoplastic elastomers, and the like. The elastomeric molded body may be formed from only one type of thermoplastic elastomer, or may be a mixture of two or more types of thermoplastic elastomers. The elastomeric molded body may further contain various types of additives (tackifier (an agent that adds tackiness), antioxidants, anti-weathering agents, ultraviolet absorbents, colorants, inorganic fillers, oils, or the like).

The fiber aggregate may, for example, be a nonwoven fabric. The materials of the fiber aggregate are not particularly limited, and various conventionally known fiber materials may be used. Examples of materials that can be used to achieve stretchability, softness and a favorable feel to the skin for the fiber aggregate include polypropylene fibers, mixed fibers formed by blending polyester fibers and polyolefin fibers, concentric composite fibers formed by covering a polyethylene terephthalate core material with a polyethylene, concentric composite fibers formed by covering a polypropylene core material with a polyethylene, or the like. The method for producing the fiber aggregate is also not particularly limited, and various conventionally known production methods can be used such as the spunbond method, the spunlace method, the thermal bond method, the meltblown method, the needle punched method, and the like.

An appropriate thickness may be selected for the substrate 21 for the sake of the flexibility of the substrate 21 with respect to the region to which the securing member is applied. For example, the lower limit of the thickness may be 50 μm, 70 μm, or 100 μm, and the upper limit of the thickness may be 500 μm, 400 μm, or 300 μm. An appropriate width may also be selected for the substrate 21 for the sake of the flexibility of the substrate 21 with respect to the region to which the securing member is applied. For example, the maximum length in the wrapping direction L of the substrate 21 may be 30 mm or greater, and 300 mm or less or 250 mm or less. Moreover, the maximum length in the widthwise direction W of the substrate 21 may be 10 mm or more, and 200 mm or less or 150 mm or less.

The first surface fasteners 30 are attached, by being glued, sewn, melt-bonded or the like, to the inner surfaces of the substrates 21. In the present embodiment, as illustrated in FIG. 1, the distal ends of the substrates 21 are not covered by the first surface fasteners 30; these distal ends function as grip sections 21a that can be easily gripped even when the first surface fasteners 30 are engaged with the loop member 14. However, the first surface fasteners 30 may also be present up to the ends of the substrates 21. Moreover, the first surface fasteners 30 may cover the entire inner surfaces of the substrates 21.

An appropriate overall shape may be selected for the first surface fasteners 30 for the sake of the stability of the securing part against shifting and the ability of the securing part to conform to the movement of the member to be secured. Examples of overall shapes for the first surface fasteners 30 include a rectangular shape, a circular shape, a rounded rectangular shape, an ellipsoidal shape, a polygonal shape, and combinations thereof.

In the present embodiment, the first surface fasteners 30 have, for example, a plurality of mushroom-shaped hooks. The plurality of hooks may be formed, for example, at a density of roughly 60 to 1,550 hooks/cm², at a density of roughly 125 to 690 hooks/cm², or at a density of roughly 248 hooks/cm². The height of each of the hooks may be roughly 30 to 1,270 μm, roughly 100 to 510 μm, or roughly 180 to 330 μm.

The diameter of each of the stems of the hooks may be, for example, roughly 76 to 635 μm, or roughly 127 to 305 μm. The head of each of the hooks has, for example, a discoid shape. The length to which the head projects out from the outer peripheral surface of the stem may be, for example, roughly 13 to 254 μm, roughly 25 to 127 μm, or roughly 120 μm. The thickness of the head may be, for example, roughly 13 to 254 μm, or roughly 25 to 127 μm. The planar shape of the head is not limited to being circular, and may instead be elliptical or polygonal. The thickness of the first surface fasteners 30 apart from the hooks, i.e., the thickness of the sheet portions of the first surface fasteners 30 supporting the hooks, may be roughly 25 to 512 μm, or roughly 64 to 254 μm.

Meanwhile, an appropriate overall shape may also be selected for the second surface fasteners 40 for the sake of the stability of the securing part against shifting and the ability of the securing part to conform to the movement of the member to be secured. Examples of overall shapes for the second surface fasteners 40 include a rectangular shape, a circular shape, a rounded rectangular shape, an ellipsoidal shape, a polygonal shape, and combinations thereof.

The second surface fasteners 40 may be present over any desired range. In the present embodiment, the second surface fasteners 40 engage with the inner surface of the dorsal side panel 17; thus, the second surface fasteners 40 may have dimensions so as to fit within the region of the dorsal side panel 17. This allows the second surface fasteners 40 to be completely covered by the dorsal side panel 17 when the ventral end 10a and the dorsal end 10b are secured. Alternatively, part of the second surface fasteners 40 may be exposed from the dorsal side panel 17 when the ventral end 10a and the dorsal end 10b are secured.

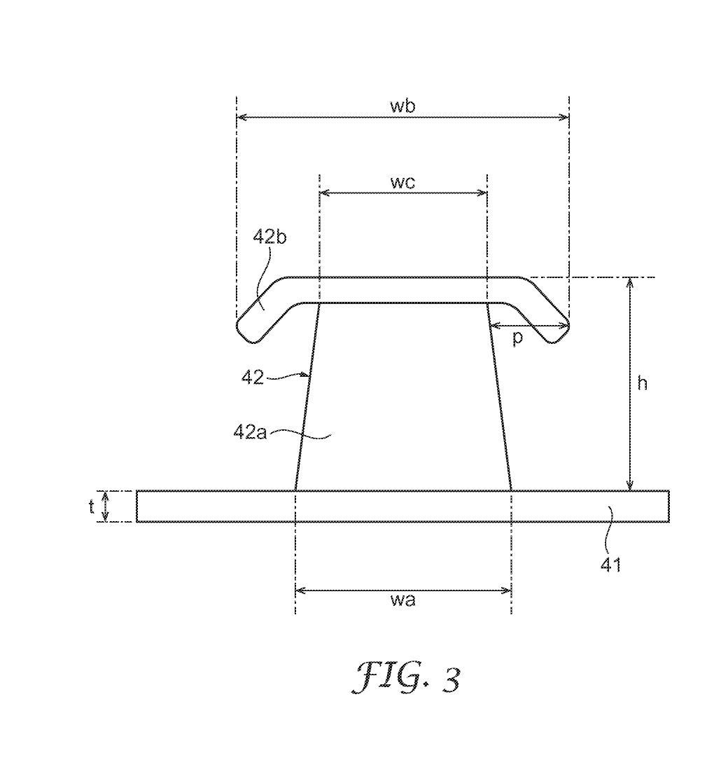

Specific embodiments of the second surface fasteners 40 are illustrated in FIGS. 3 to 20.

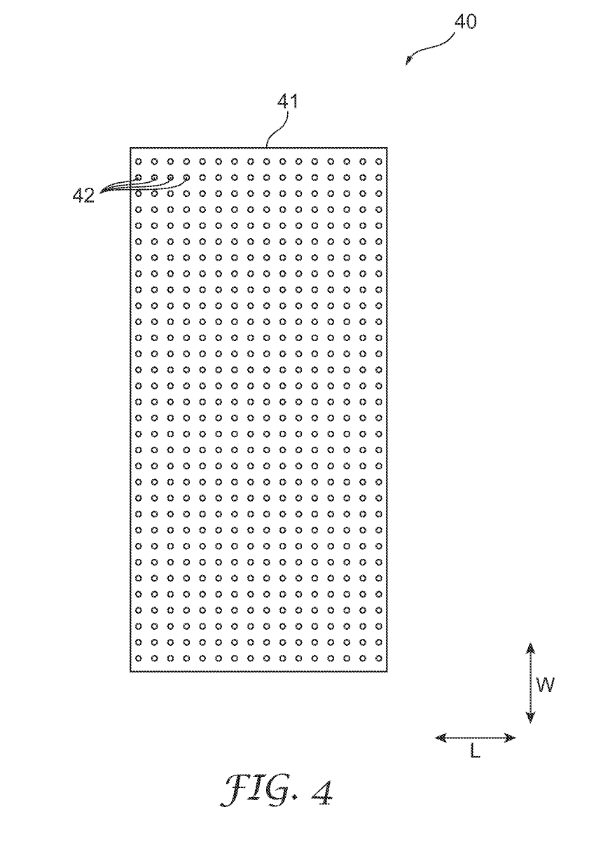

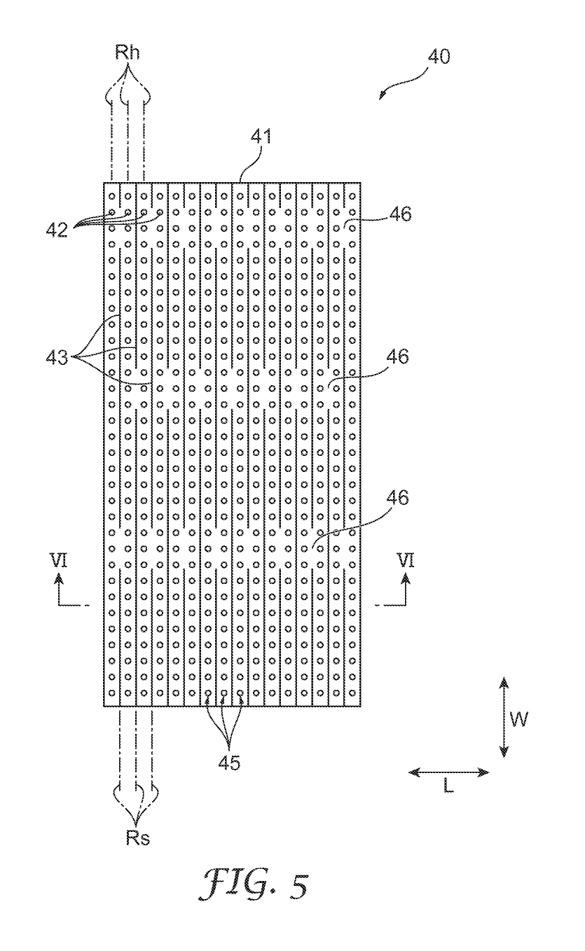

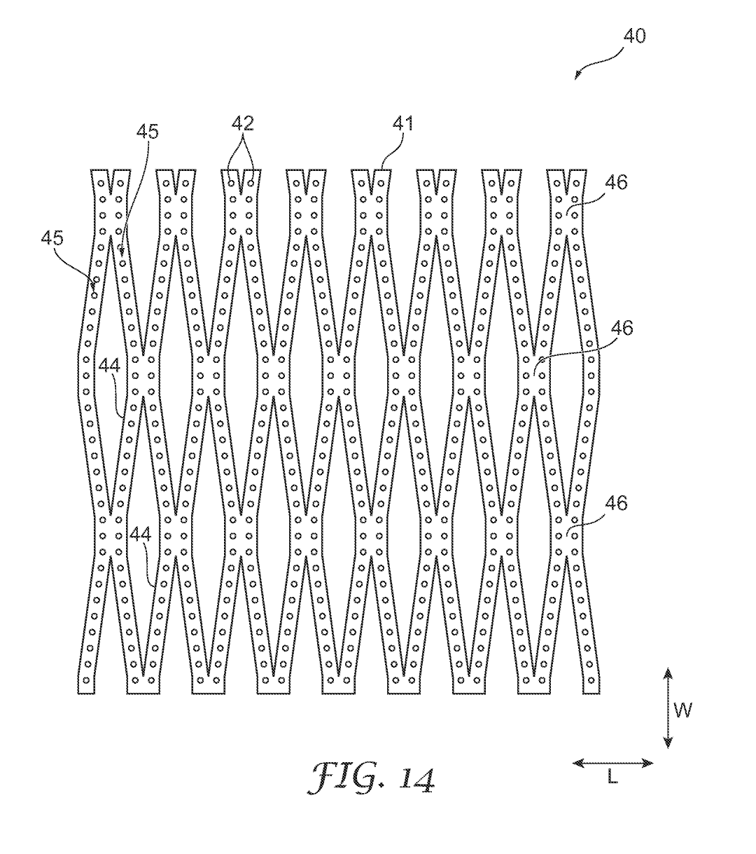

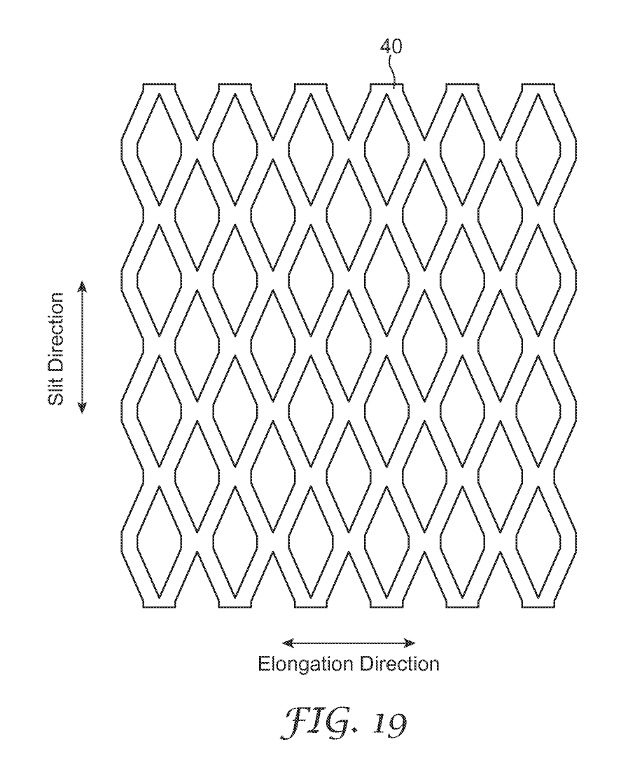

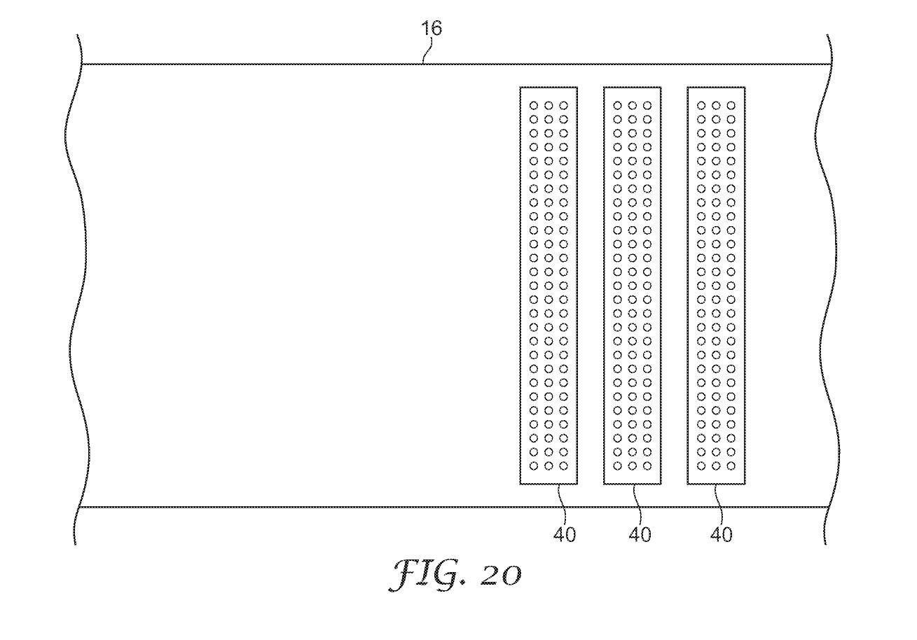

FIG. 3 illustrates an example of a hook. FIG. 4 is an example of a second surface fastener 40 in which neither grooves nor through-holes are formed. FIGS. 5 to 18 illustrate examples of second surface fasteners 40 in which grooves or through-holes are formed. More specifically, FIGS. 5 to 13 illustrate examples of second surface fasteners 40 in which a plurality of slit-shaped grooves or through-holes are formed, and FIGS. 14 to 18 illustrates examples of second surface fasteners 40 in which non-slit-shaped through-holes (openings) are formed. In the present specification, elongated hook member regions sandwiched between adjacent grooves or through-holes may be referred to as strands. Note that, from FIG. 4 on, the hooks 42 illustrated in FIG. 3 are illustrated in a simplified form. FIG. 19 illustrates one example of a method of forming openings. FIG. 20 illustrates an example in which spaces are provided among a plurality of second surface fasteners 40.

As illustrated in FIG. 4, hooks having neither slit-shaped grooves nor through-holes nor openings may be used for the second surface fasteners 40. If such grooves, through-holes, and openings are not formed, the thickness of the second surface fasteners 40 will generally be 60 to 380 μm. In some embodiments, for the sake of the stability of the securing part against shifting and the ability of the securing part to conform to movement of the member to be secured, the lower limit of the thickness can be 90 μm or 115 μm, and the upper limit of the thickness can be 350 μm or 320 μm. In the present specification, a surface fastener whose thickness falls within the range of 60 to 380 μm is also referred to as a “low-profile fastener”. Using low-profile fasteners of low stiffness as the second surface fasteners 40 allows the second surface fasteners 40 themselves to flexibly deform, minimizing the stiffness of the regions in which the second surface fasteners 40 are provided, even if grooves, through-holes, or openings are not provided.

The second surface fastener 40 includes a sheet-shaped base 41 and a plurality of the hooks 42 provided on the surface of the base 41. In the present embodiment, as illustrated in FIG. 3, the hook 42 is provided with a stem 42a extending from the base 41 and a head 42b formed at the distal end of the stem 42a, and has an overall mushroom-like shape. Note that the head 42b of the hook 42 may have any type of shape that offers satisfactory engagement strength; apart from the above-mentioned mushroom shape, for example, a hook shape, T-shape, or J-shape is possible.

If the second surface fastener 40 illustrated in FIG. 4 is used, the thickness and height of the second surface fastener 40 illustrated in FIG. 3 can be set, for example, within the following ranges for the sake of the stability of the securing part against shifting and the ability of the securing part to conform to movement of the member to be secured.

The lower limit of the thickness t of the base 41 may be 20 μm, 30 μm, or 35 μm, and the upper limit of the thickness t may be 80 μm, 70 μm, or 60 μm.

The lower limit of the height h of the hook 42 may be 40 μm, 60 μm, or 80 μm, and the upper limit of the height h may be 300 μm, 280 μm, or 260 μm.

There is no particular limitation upon the maximum width wa of the base of the stem 42a or upon the distal width wc of the stem 42a as long as the desired engagement strength can be obtained. Taking the mushroom-shaped hook member as illustrated in FIG. 3 as an example, the lower limit of the maximum width wa of the base of the stem 42a may be 70 μm or 100 μm, and the upper limit of the maximum width wa may be 250 μm, 200 μm, or 190 μm. The lower limit of the distal width wc of the stem 42a may be 50 μm or 80 μm, and the upper limit of the width wc may be 200 μm, 195 μm, or 185 μm.

Moreover, the maximum width wb of the head 42b can be determined, as appropriate, for the sake of engagement strength. As illustrated in FIG. 3, the maximum width wb of the head 42b is preferably greater than the maximum width wa of the base of the stem 42a, and the maximum ratio of the maximum width wb of the head 42b to the maximum width wa of the base of the stem 42a may be 1.01:1, 2:1, or 3:1. The lower limit of the maximum width wb may be 70 μm or 100 μm, and the upper limit of the maximum width wb may be 350 μm or 340 μm. The lower limit of the projection distance p to which the head 42b projects from the distal end of the stem 42a may be 5 μm or 10 μm, and the upper limit of the projection distance p may be 90 μm, 85 μm, 80 μm, or 75 μm.

Based on the thickness of the base 41 and the height of the hook 42 as described above, the lower limit of the overall thickness of the second surface fasteners 40 may be 60 μm, 90 μm, or 115 μm, and the upper limit of the thickness may be 380 μm, 350 μm, or 320 μm. Consideration is to be given to the flexibility of the regions to which the second surface fasteners 40 are attached in settling upon the overall thickness of the second surface fasteners 40. Moreover, in one embodiment, for example, the lower limit of a ratio of the thickness of the second surface fasteners 40 to the thickness of the regions on which the second surface fasteners 40 are provided (region thickness/surface fastener thickness) can be 0.02, 0.03, or 0.05, and the upper limit of the ratio can be 7.6, 5, or 3.2. In this case, the thickness of the regions on which the second surface fasteners 40 are provided is the thickness of the substrate, and, in the present embodiment, is the thickness of the substrate of the ventral side panel 16.

Meanwhile, if grooves, through-holes, or openings are formed in the second surface fasteners 40, as illustrated in FIGS. 5 to 18, low-profile fasteners may be used as the second surface fasteners 40, or surface fasteners having a thickness of 380 μm or more may be used, as in the case of the first surface fasteners 30.

Here, in the context of the present specification, the term “groove” refers to a state in which cuts are provided in the surface of the base 41 on the side of the second surface fasteners 40 on which the hooks 42 are present, these cuts not penetrating through to the surface of the base 41 opposite that on which the hooks 42 are present. In the present embodiment, the term “grooves” refers to slit-shaped grooves 43b, but the range encompassed by this term is not limited to thereto. For example, the concept of “grooves” also encompasses depressions that extend not linearly but in a two-dimensional (planar) shape.

Moreover, in the context of the present specification, the term “through-holes” refers to a state in which holes or openings provided in the second surface fasteners 40 penetrate from the surface of the base 41 on the hooks 42 side through to the surface of the base 41 opposite the hooks 42 side. In the present embodiment, the term “through-holes” refers to slit-shaped through-holes 43a, as well as through-holes in a shape other than a slit-shape (i.e., the openings 44), but the range encompassed by this term is not limited. For example, the concept of “through-holes” encompasses dot-shaped holes provided in the second surface fasteners 40, as well as penetrating slits having a non-linear wave shape, chevron shape, or irregular shape.

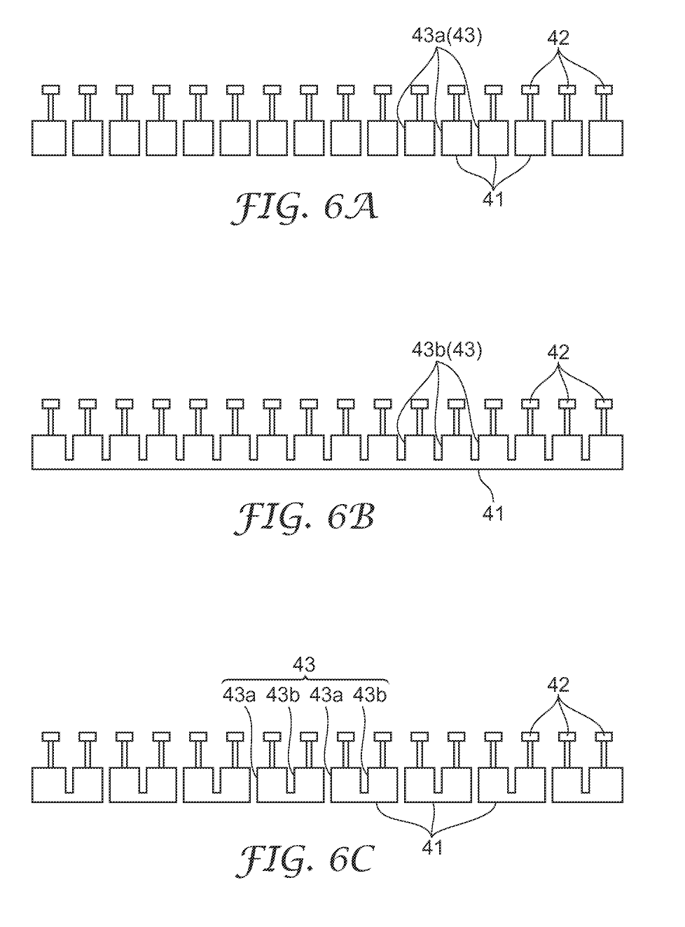

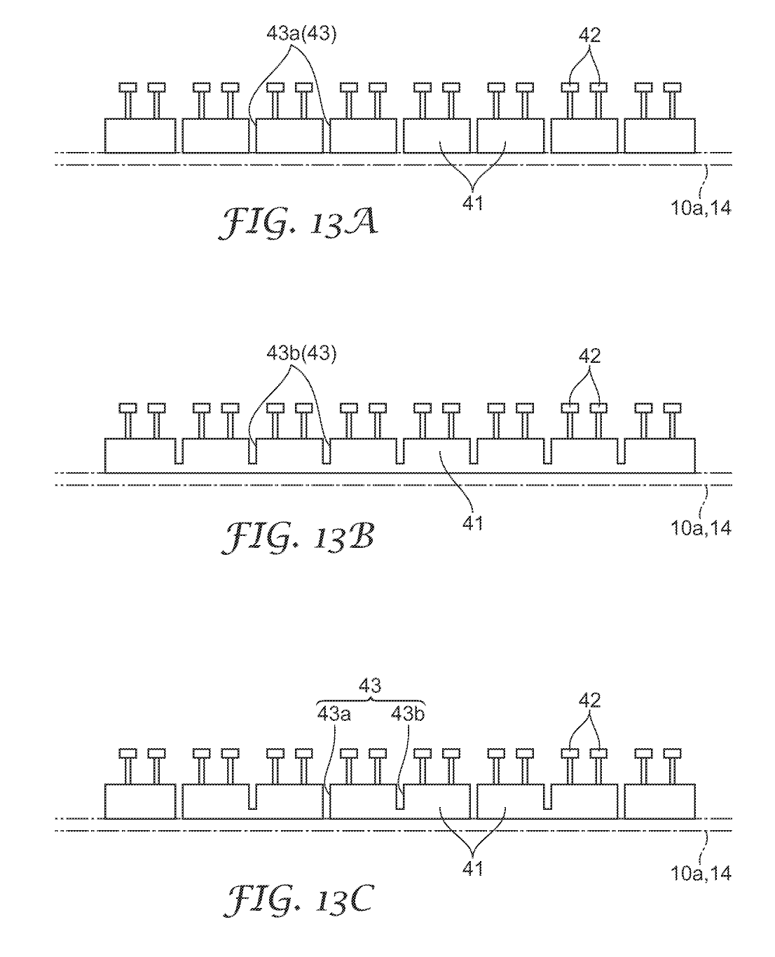

As illustrated in FIG. 6B, the slit-shaped grooves 43b are formed on only one surface of the second surface fasteners 40, namely, the surface of the base 41 on the hooks 42 side, and do not extend through to the surface opposite the hooks 42 side. By contrast, the slit-shaped through-holes 43a illustrated in FIG. 6A and the openings 44 illustrated in FIGS. 14 to 18 are openings that are formed extending from one side of the surface fastener through to the other surface. As illustrated in FIGS. 6C, 13 C, and 15, both grooves and through-holes may be present in the second surface fasteners 40.

The grooves and through-holes 43 can be formed using any conventionally used technique. For example, the slit-shaped grooves 43b and the through-holes 43a can be formed via blade or laser cutting, or the like, so that the grooves 43b extend from the surface of the base 41 on the hooks 42 side to a certain thickness of the base 41, and the through-holes 43a extend from the surface of the base 41 on the hooks 42 side to the surface opposite the hooks 42 side. Meanwhile, openings 44, which are another example of through-holes, can be formed, for example, by expanding the second surface fasteners 40 in which the slit-shaped through-holes 43a are formed in a direction orthogonal to the rows of slits. Moreover, the openings 44 may also be formed without expanding the second surface fasteners 40 by making cutouts of a desired shape in the second surface fasteners 40. Note that the openings 44 may also be formed by forming the slit-shaped through-holes 43a and applying the second surface fasteners 40 to the substrate, followed by expanding the slit-shaped through-holes 43a along with the substrate. Examples of means of expanding the second surface fasteners 40 include devices such as tenters or rollers, or manual labor. There is no limitation upon the elongation rate of the second surface fasteners 40 when the openings are formed; the upper limit thereof may be, for example, 50%, 100%, 150%, 200%, 300%, 400%, or 600%. In this context, the elongation rate refers to the degree to which the second surface fasteners 40 are elongated with respect to the original (initial) length in the direction in which the through-holes are expanded to form the openings (i.e., the elongation direction in FIG. 19). In other words, an elongation rate of 50% indicates that the length of the second surface fasteners 40 is 1.5 times the initial length, an elongation rate of 100% indicates that the length of the second surface fasteners 40 is two times the initial length, and an elongation rate of 200% indicates that the length of the second surface fasteners 40 is three times the initial length.

If low-profile fasteners having the overall thickness described above are used as the second surface fasteners 40, the stiffness of the second surface fasteners 40 will be reduced, allowing the second surface fasteners 40 themselves to flexibly deform. Moreover, the stiffness of the second surface fasteners 40 can also be reduced by forming grooves or through-holes 43 in the second surface fasteners 40, thereby allowing the second surface fasteners 40 themselves to flexibly deform. Thus, if the second surface fasteners 40 are attached to the regions having flexible, the overall flexibility of the regions can be maintained. As a result, the flexibility of the securing part increases, and the securing part is capable of conforming to the movement of the member to be secured, keeping the securing members 20 from detaching from the loop member 14 or the ventral end 10a when the ventral end 10a and the dorsal end 10b are secured. In addition, the second surface fasteners 40 formed from low-profile fasteners or the second surface fasteners 40 having grooves or through-holes 43 are soft, eliminating uncomfortably bulky sensations and reducing the degree of wearer skin irritation experienced when the second surface fasteners 40 are attached to an absorbent article or a garment. The utilization of such second surface fasteners 40 thus increases the user-friendliness of the absorbent article.

In the example illustrated in FIG. 5, slit-shaped grooves or through-hole rows Rs (also referred to as slit rows Rs) are formed between hook rows Rh along the widthwise direction W of the second surface fasteners 40 so that each hook row Rh is sandwiched between adjacent slit rows Rs. Accordingly, in the present example, there is one hook 42 in the widthwise direction (i.e., the wrapping direction L of the second surface fastener 40) of a strand 45 constituted by a hook row Rh. In other words, the width of each strand 45 is equivalent to one hook 42 (more precisely, one hook 42 and a fixed length of the base 41 extending from the hook 42 ). In each of the slit rows Rs, a plurality of uniformly spaced slit-shaped grooves or through-holes 43 are formed; in the embodiments illustrated in FIGS. 5 to 11 and 14 to 18, the regions corresponding to the spacing (the regions where the base 41 is left unmodified) are referred to as bridge sections 46. In the example illustrated in FIG. 5, the bridge sections 46 are arranged in a staggered manner.

As illustrated in FIG. 6, regarding the slit-shaped grooves or through-holes 43, all of the slits in the example of FIG. 6A penetrate the base 41, thereby forming the slit-shaped through-holes 43a. Moreover, in the example of FIG. 6B, all of the slits are grooves, and slit-shaped grooves 43b, having a constant depth from the surface of the base 41 on which the hooks 42 are present, are formed. Moreover, as with the example of FIG. 6C, the slit-shaped through-holes 43a penetrating the base 41 and the slit-shaped grooves 43b having a groove shape may also be arranged in an alternating manner. In a case in which the slit 43 is a groove, the lower limit of the ratio of the depth of the slit 43 with respect to the thickness of the base 41 may be 0.4, and the upper limit thereof may be 0.9.

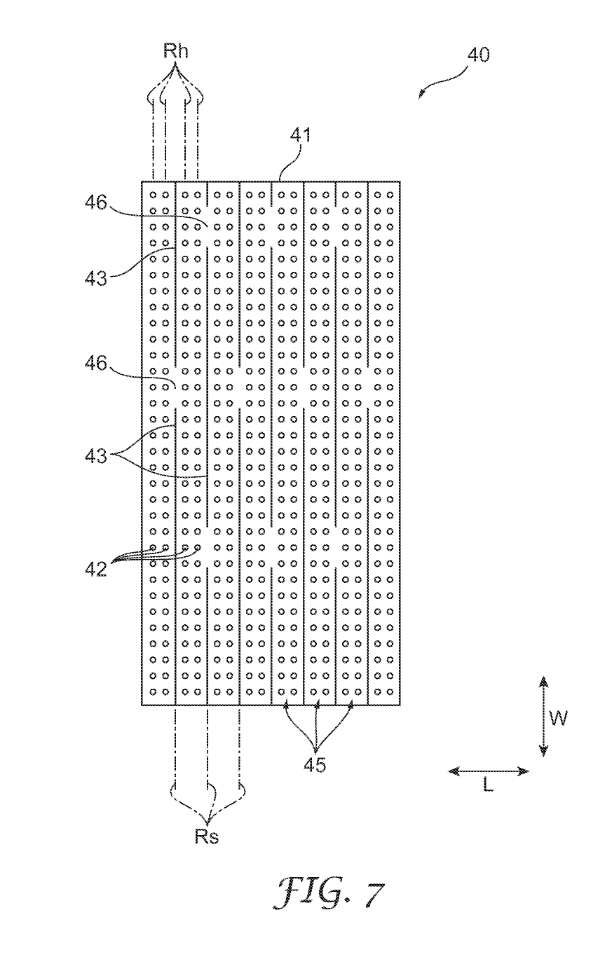

There is no limitation upon the width of the strands 45. For example, as illustrated in FIG. 7, the width of the strand 45 may also be equivalent to a portion containing two hooks 42, or may be equivalent to a portion containing three or more hooks 42. The maximum width of the strand 45 may also be equivalent to a portion containing ten hooks 42.

There is no limitation upon the spacing between the slit rows Rs provided in the second surface fasteners 40. For example, the minimum number of slit rows Rs per 1 cm in a direction orthogonal to the slit rows Rs may be 1, and the maximum number may be 10.

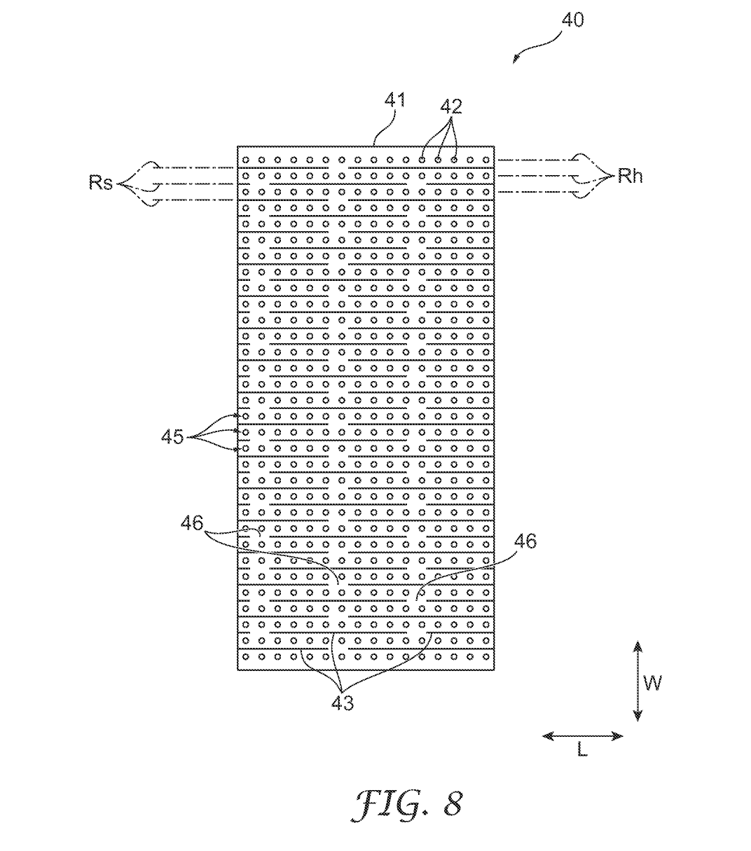

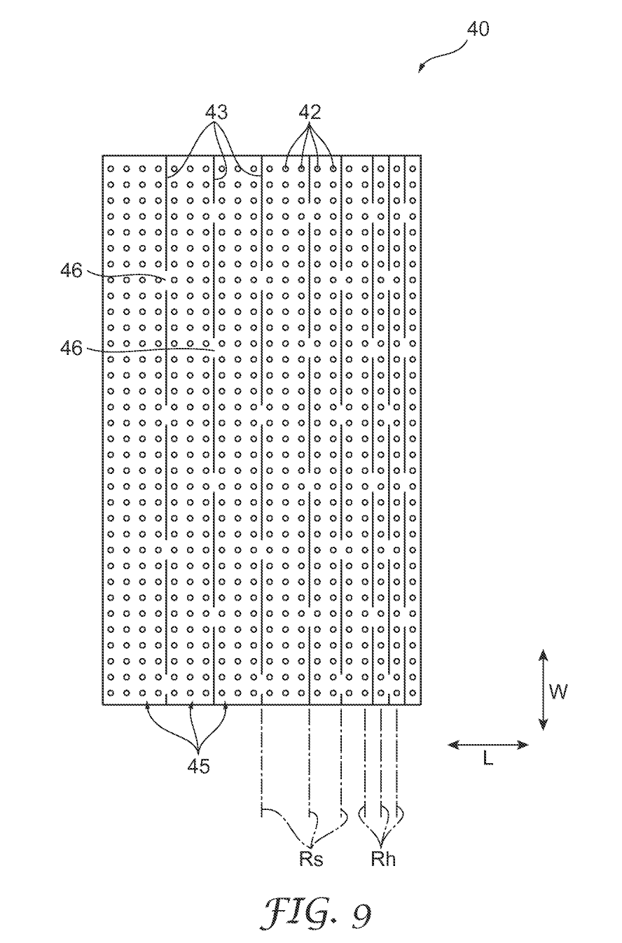

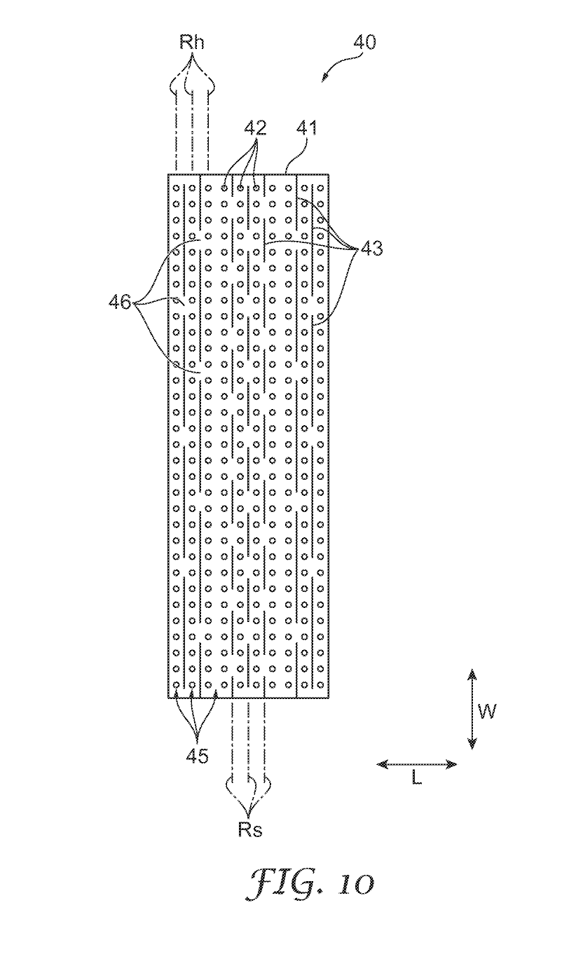

The direction in which the slit-shaped grooves or through-holes 43 extend is also not limited. For example, as illustrated in FIG. 8, each of the slit rows Rs may extend along the wrapping direction L. Alternatively, each of the slit rows Rs may be slanted at a desired angle θ (0°<0<90°) with respect to the wrapping direction L or the widthwise direction W. The strands 45 need not be uniform in width. For example, as illustrated in FIG. 9, the slits 43 may be formed so that the strands 45 gradually narrow toward one end. Alternatively, as illustrated in FIG. 10, a mixture of strands 45 containing only a single hook row Rh and strands 45 containing two hook rows Rh may be used.

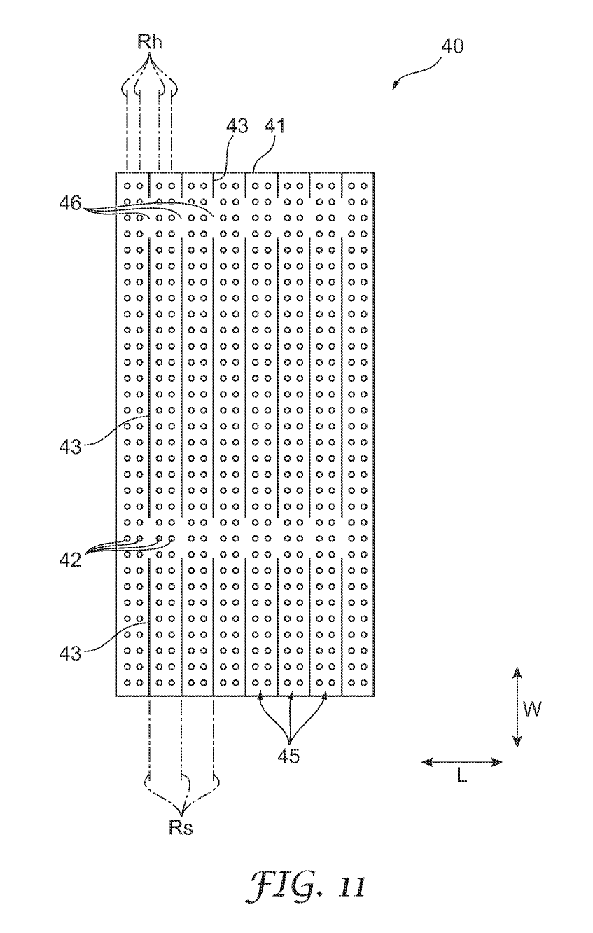

The bridge sections 46 are not limited to being arranged in a staggered manner as illustrated in FIG. 5. For example, as illustrated in FIG. 11, any desired adjacent bridge sections 46 may be arranged along a direction orthogonal to the slit rows Rs (in FIG. 11, direction L).

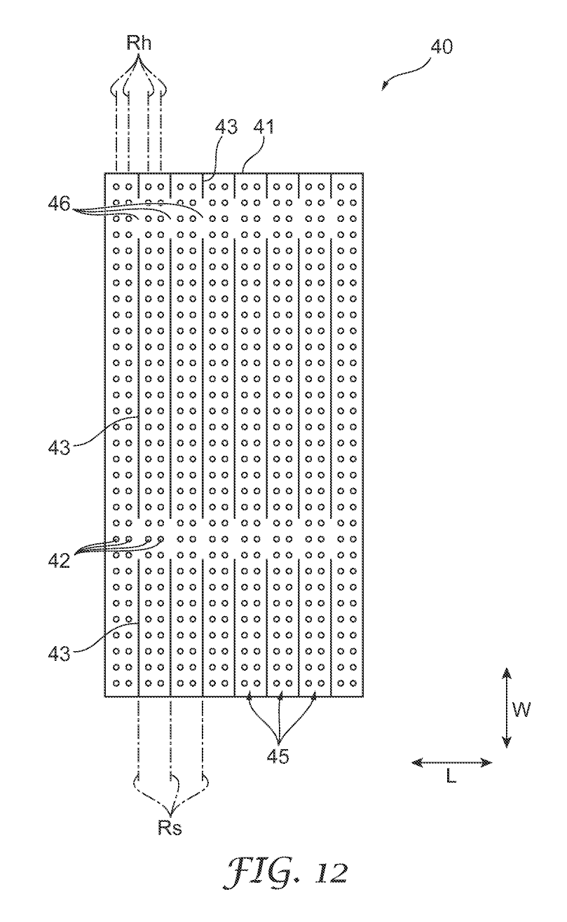

As illustrated in FIG. 12, a single slit-shaped groove or through-hole 43 may extend from one side of the second surface fastener 40 to the opposite side without interruption. In this instance, no bridge sections 46 are present on the second surface fasteners 40. In this instance, the aspect of the slit rows Rs is such that all of the slits penetrate the base 41 to form slit-shaped through-holes 43a, as illustrated in FIG. 13A, or all of the slits may be grooves and slit-shaped grooves 43b, having a constant depth from the surface of the base 41 on which the hooks 42 are present, may be formed as illustrated in FIG. 13B. Moreover, as with the example of FIG. 13C, the slit-shaped through-holes 43a penetrating the base 41 and the slit-shaped grooves 43b having a groove shape may also be arranged in an alternating manner. In a case in which the slits 43 are grooves, the lower limit of the ratio of the depth of the slits 43 to the thickness of the base 41 may be 0.4, and the upper limit of the ratio may be 0.9.

The length of the slit-shaped grooves or through-holes 43 may be set as desired. For example, the lower limit of the length of each slit-shaped groove or through-hole 43 may be 8 mm, 10 mm, or 12 mm. Alternatively, the slit-shaped grooves or through-holes 43 can be stipulated by the ratio of the total length of the slit-shaped grooves or through-holes 43 within the slit rows Rs to the length of the second surface fasteners 40 along the slit rows Rs (total length of slit-shaped grooves or through-holes/length of slit rows Rs). For example, the ratio can be at least 40% or more, or 50% or more.

The length of the bridge sections 46 may also be set as desired. For example, the lower limit of the length of the bridge sections 46 may be 0.25 mm, 0.5 mm, or 0.75 mm, and the upper limit of the length may be 10 mm, 15 mm, or 20 mm.

FIG. 14 illustrates a second surface fastener 40 in which openings 44 are provided. The openings 44 illustrated in FIG. 14 are formed by expanding slits extending in the widthwise direction W of the second surface fastener 40 in the wrapping direction L. In this example, the width of each strand 45 is equivalent to the portion of one hook 42, and the openings 44 and bridge sections 46 are arranged in a staggered manner, respectively.

In the example of FIG. 14, the openings 44 have a roughly rhombic shape, but the shape of the openings is not limited thereto. For example, the openings may be hexagonal; if such openings are arranged in a staggered manner, the second surface fastener 40 can be considered to have a honeycomb structure. Alternatively, the openings may be circular, elliptical, rectangular, star-shaped, wave-shaped, or otherwise polygonal shaped.

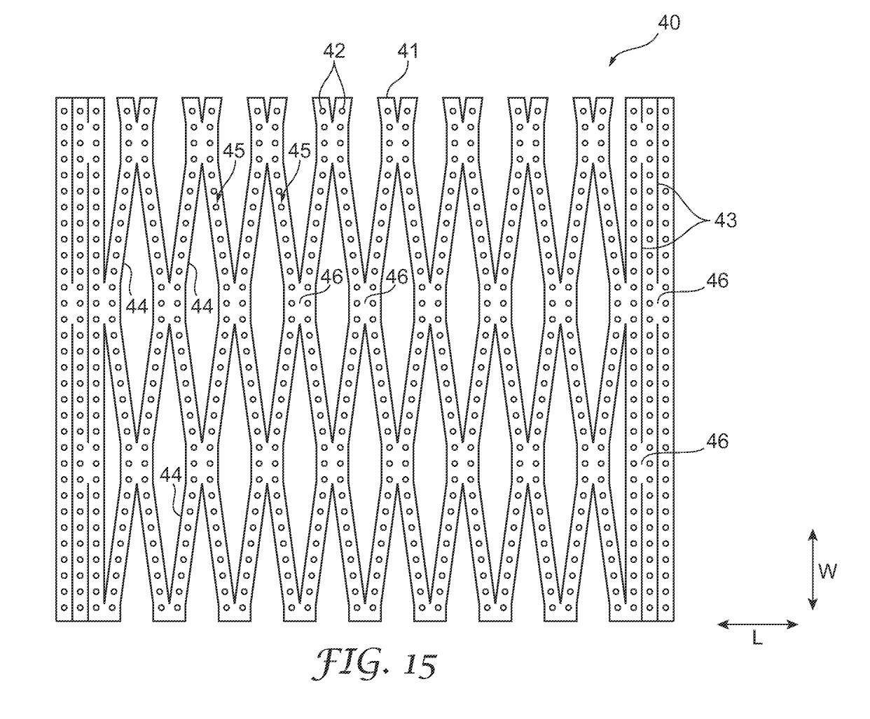

As illustrated in FIG. 15, a second surface fastener 40 in which both openings 44 and slit-shaped grooves or through-holes 43 are present may be prepared by expanding only some of the plurality of slits. In the present example, the openings 44 are provided in the center of the second surface fastener 40, and the slit-shaped grooves or through-holes 43 are provided near both ends thereof, but the slit-shaped grooves or through-holes 43 and openings 44 are not limited to being so positioned. For example, the slit-shaped grooves or through-holes 43 may be provided in the center, and the openings 44 may be provided near both ends.

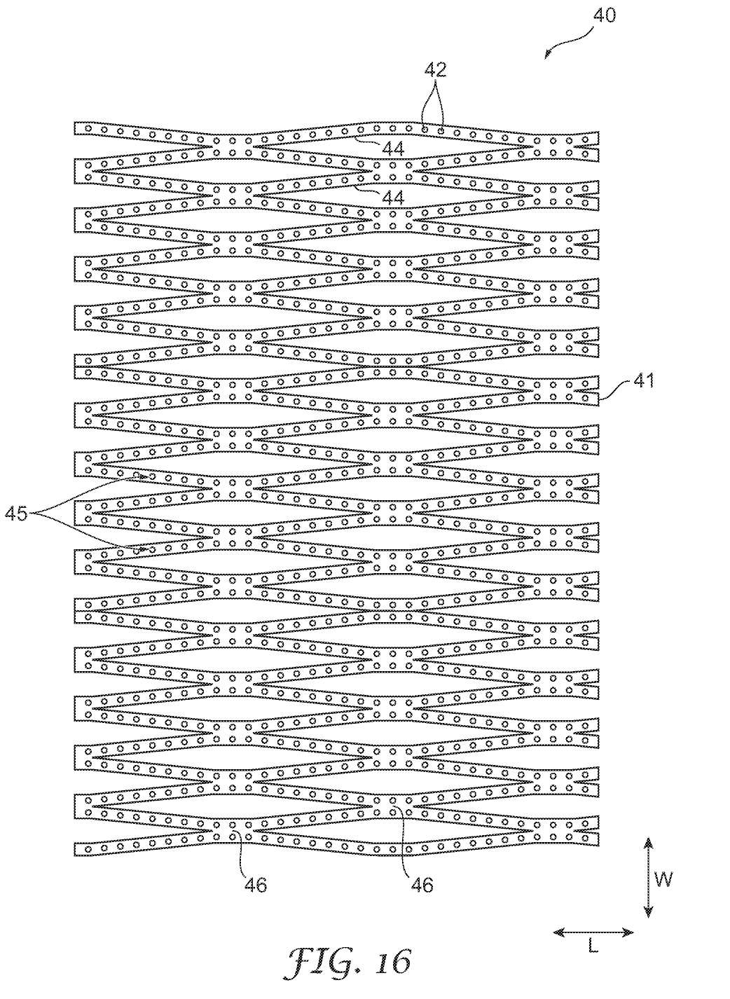

The openings 44 illustrated in FIG. 16 are formed by expanding slits extending in the direction L. In this way, the extension direction of the slit-shaped through-holes 43, which become the basis for the openings, is not limited.

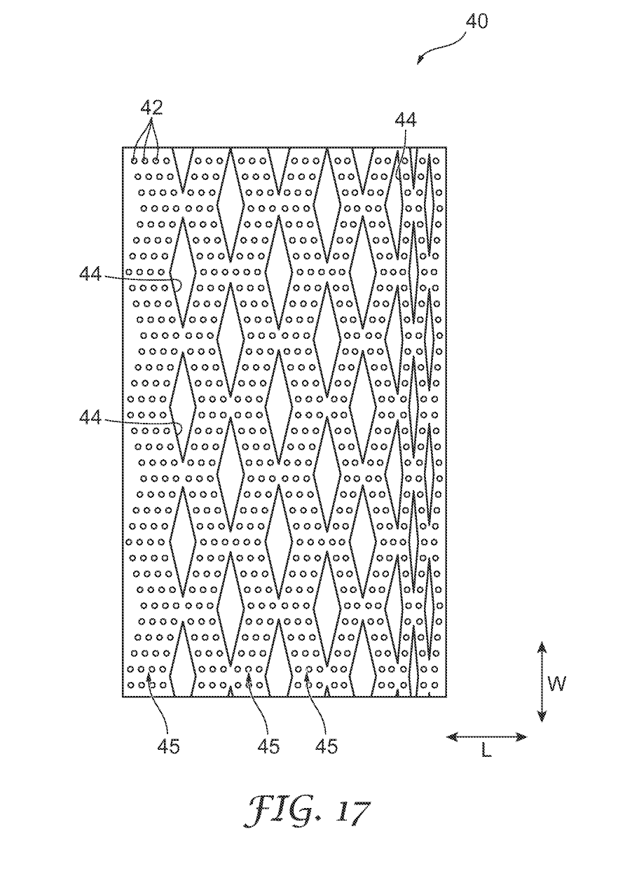

Even in a case in which the openings 44 are utilized as through-holes, similar to a case of slit-shaped grooves or through-holes 43, the width of the strands 45 is not limited. For example, the width of each strand 45 may be equivalent to a portion containing two hooks 42, and the maximum value of the width may be equivalent to a portion containing ten hooks 42. The strands 45 need not be uniform in width; for example, as illustrated in FIG. 17, the openings 44 may be formed so that the strands 45 gradually narrow toward one end.

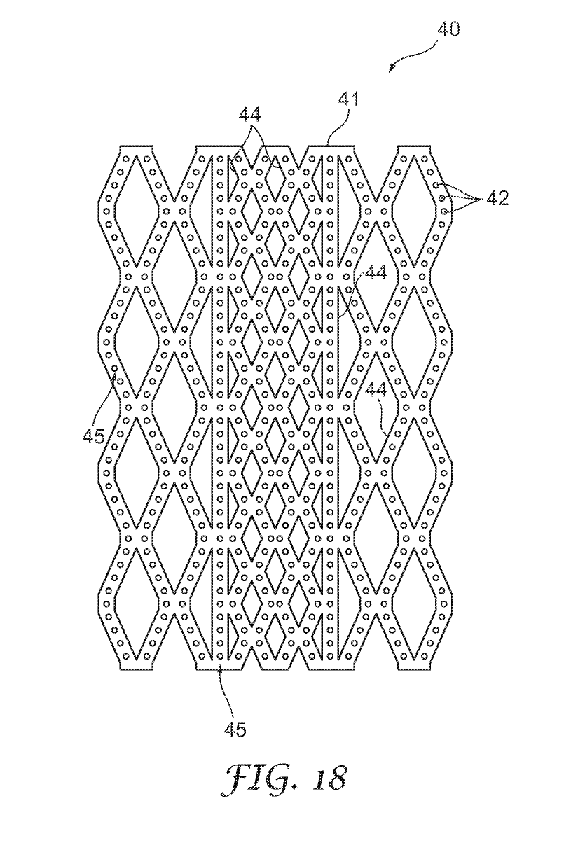

The size of the openings 44 is not required to be uniform. For example, as illustrated in FIG. 18 and the above FIG. 17, the size of the openings 44 may be non-uniform. Moreover, the openings 44 may also be of various shapes, as illustrated in FIG. 18.

Regarding a second surface fastener 40 in which openings 44 are formed, the bridge sections 46 do not have to be arranged in a staggered manner. For example, the bridge sections 46 may be arranged in a grid-like manner, as in the example illustrated in FIG. 11.

If a second surface fastener 40 in which openings 44 are formed is attached to a substrate using a bonding agent or adhesive agent, the bonding agent or adhesive agent may be exposed from the openings 44.

The density of the hooks 42 in the hook member, attached to the substrate, in which grooves or through-holes are formed will now be described. The hook density is determined from the “total number of hooks in the hook member/total surface area of the base 41 and openings 44 in the hook member.” Note that if a plurality of second surface fasteners 40 are disposed on the substrate with spaces therebetween, the area of the spaces among the plurality of second surface fasteners 40 is not included in the aforementioned total surface area. The lower limit of the density of the hooks 42 on the substrate 21 can be, for example, approximately 31 hooks/cm², or approximately 39 hooks/cm². Moreover, the upper limit of the density may be, for example, approximately 1,550 hooks/cm², or approximately 1,240 hooks/cm². For example, the density may be approximately 465 hooks/cm².

If the openings 44 are formed by expanding a second surface fastener 40 in which slit-shaped through-holes 43 are formed, and the second surface fastener 40 is attached to a substrate while maintaining the shapes of the openings 44 so formed, the initial density of the hooks 42 on the second surface fastener 40 before expansion may be set as follows. Specifically, the lower limit of the initial density may, for example, be 50 hooks/cm², 70 hooks/cm², or 100 hooks/cm², and the upper limit thereof may, for example, be 1,550 hooks/cm². Note that the initial density of the hooks 42 is determined from the “total number of hooks in the hook member / total surface area of the base 41 in the hook member before expansion.” Note that if a plurality of second surface fasteners 40 are disposed on a substrate with spaces therebetween, the initial densities of the respective second surface fasteners 40 may be in the range set forth above.

Moreover, the lower limit of the proportion of the total surface area of the heads 42b to the overall surface area of the second surface fastener 40 when the second surface fastener 40 is attached to a substrate (the relative density of the heads) may be, for example, 5% or 10%. The upper limit of the proportion may be, for example, 24%, 30%, or 40%. Note that if a plurality of second surface fasteners 40 are disposed on a substrate, the proportions of the respective second surface fasteners 40 may be in the range set forth above.

If a plurality of second surface fasteners 40 are provided on the substrate (ventral side panel 16) with spaces therebetween, as illustrated in FIG. 20, low-profile fasteners may be used as the second surface fasteners 40, or surface fasteners having a thickness of 380 μm or more may be used, as in the case of the first surface fasteners 30. In this instance, the spaces play a role equivalent to the slit-shaped grooves or through-holes, or openings described above, eliminating the need to form slit-shaped grooves or through-holes, or openings in the respective second surface fasteners 40. Of course, slit-shaped grooves or through-holes, or openings may be formed in some or all of the second surface fasteners 40. If a plurality of second surface fasteners 40 are provided on the ventral side panel 16, the respective second surface fasteners 40 may have an identical or different shape.

The second surface fasteners 40 are formed, for example, from a thermoplastic resin. First, a sheet material having a plurality of columnar bodies, which establish the original form of a plurality of hooks, lined up on the surface, is formed through extrusion molding using a template or die having a multiplicity of through holes. Next, the distal end of each of the columnar bodies is heated and, for example, crushed into a circular plate shape to form each of the hooks 42 having the head 42b, thereby obtaining the second surface fastener 40. Examples of thermoplastic resins include polyolefins such as polyethylene and polypropylene, polyethylene terephthalate, nylon and other polyamides, poly (styrene-acrylonitrile), poly (acrylonitrile-butadiene-styrene), plasticized vinyl chloride, polyesters, and the like. One type of these thermoplastic resins may be used singly, or a polymer blend containing a mixture of two or more types of thermoplastic resins may be used. A polyethylene-polypropylene copolymer may also be used.

By attaching the second surface fastener 40 described using FIGS. 3 to 20 to a substrate, the bending stiffness of the securing member provided with the second surface fastener 40 can be reduced. As discussed above, in the securing member provided with the second surface fastener 40, the flexibility of the second surface fastener 40 is indicated by determining the ratio between the bending stiffness Sa of the laminated section formed by the substrate and the second surface fastener 40 laminated on the substrate and the bending stiffness Sb of a comparison member constituted by a laminate formed from the same substrate and a piece of cellophane adhesive tape (comparison tape) as specified in JIS Z 1522. When comparing the bending stiffness of the securing member having the second surface fastener 40 and the bending stiffness of the comparison member, the same conditions are used for both substrates. In the following description, the laminated section of the securing member used to measure bending stiffness and the laminate constituting the comparison member may also be collectively referred to as a test strip. In the test strip obtained from the laminated section of the securing member, the second surface fastener 40 covers the entirety of one surface of the substrate. In the laminate constituting the comparison member, the comparison tape covers the entirety of one surface of the substrate.

A method of obtaining a test strip for measuring bending stiffness from the diaper 1 illustrated in FIG. 1 will be described as one example. First, a laminated section having the substrate and the second surface fastener 40 laminated on the substrate (a laminate formed from the substrate and the second surface fastener 40 of the securing member) is obtained by cutting a section out of the ventral side panel 16 upon which the second surface fastener 40 is laminated.

Meanwhile, the comparison member is obtained by cutting a substrate out of a portion of the ventral side panel 16 upon which the second surface fastener 40 is not laminated and by laminating a piece of comparison tape upon the substrate to form a laminate. The comparison tape is applied to the same surface, out of the outer surface and the inner surface of the substrate, upon which the second surface fastener 40 is laminated. The dimensions of the test strips may be determined with consideration to the type of measuring apparatus or the dimensions of test strips that can be obtained. The test strips are prepared so that the second surface fastener 40 or comparison tape covers the entirety of one surface of the substrate.

Next, a method of measuring bending stiffness will be described. In the context of the present specification, the term “bending stiffness” signifies bending stiffness as measured via a cantilevered bending test using a Gurley testing apparatus according to the method of TAPPIT 543 om-11. The bending stiffness measured according to this method is sometimes referred to as “Gurley stiffness” by persons skilled in the art. The bending test is performed in an environment at a temperature of 23° C. and a relative humidity of 50%.

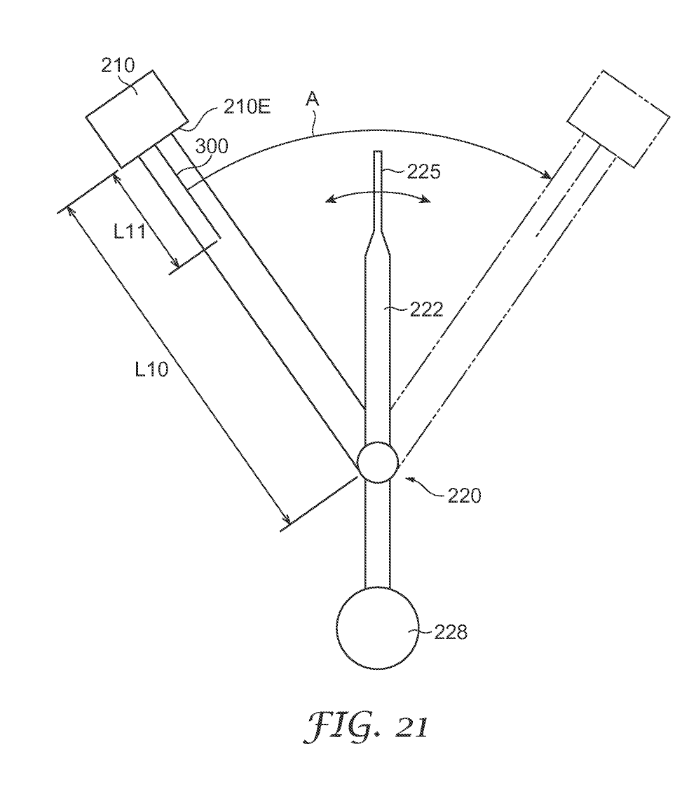

FIG. 21 is an illustration of a bending test method for measuring bending stiffness (Gurley stiffness). As illustrated in FIG. 21, one end of the test strip (laminated section of the securing member, or comparison member) is gripped between two clamps 210. The clamps 210 rotate around a rotating shaft 220 to move a test strip 300 in a circumferential direction A around the rotating shaft 220. A plate-shaped member 225 is provided on one end of a pendulum shaft 222 rotatably provided around the rotating shaft (support point) 220, and a weight 228 is attached to the shaft 222. The mass and attachment position of the weight 228 are selected, as appropriate, according to the degree of the bending stiffness of the test strip 300, or the like.

When the test strip 300 rotates at a constant speed in the circumferential direction A (i.e., clockwise), a free end of the test strip 300 comes into contact with the plate-shaped member 225, causing the test strip 300 to bend. As the test strip 300 continues to move, the plate-shaped member 225 is released from the test strip 300 after being pushed by the test strip 300 and rotating to a certain degree. The bending stiffness is obtained based on the maximum load placed upon the plate-shaped member 225 in this process. The test strip 300 is then moved in the opposite direction (i.e., counterclockwise), allowing bending stiffness when the test strip 300 is bent to the opposite side to be measured. The movement speed of the test strip is set to one cycle of reciprocation per minute. The bending stiffness is determined as the average value of two measured values taken as the test strip 300 moves back and forth in the circumferential direction.

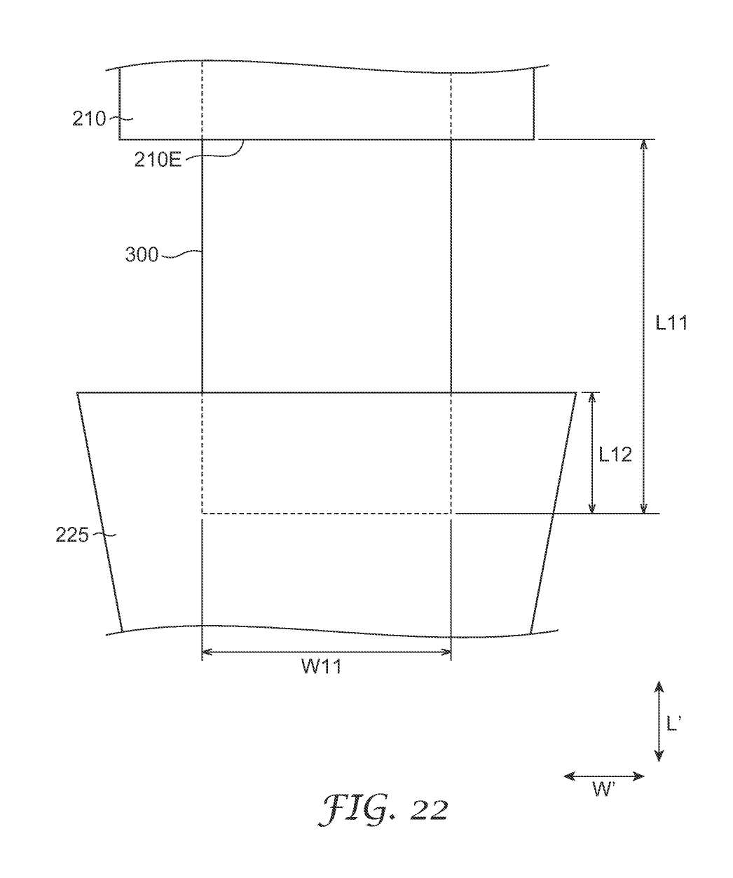

FIG. 22 illustrates the position at which the test strip 300 is anchored for the bending test. The test strip 300 has a strip-like shape. The front and rear sides of the gripped test strip 300 can be oriented as desired. The total length of the test strip 300 along a direction L′ from the clamps 210 toward the rotating shaft 220 is set to, for example, 25.4±0.4 mm (0.25± 1/64 inches). The length in the direction L′ of a portion of the test strip 300 gripped by the clamps 210 is set to 6.35 mm, and a portion having a length L12 from the free end is placed so as to come into contact with the plate-shaped member 225. The length L12 is set to 6.35 mm (0.25 inches). Meanwhile, the length (width) W11 in a direction W′ orthogonal to the direction L′ is 25.4±0.4 mm (1± 1/64 inches) or 12.7±0.4 mm (0.5± 1/64 inches). Note that the direction L′ may not necessarily be the same as the wrapping direction L, nor may the direction W′ necessarily be the same as the widthwise direction W.

As discussed above, the relationship Sa/Sb<2.0 holds between the bending stiffness Sa of the laminated section formed by the substrate and the second surface fastener 40 laminated on the substrate, and the bending stiffness Sb of the comparison member. Alternatively, Sa/Sb may be 1.7 or less, 1.6 or less, 1.5 or less, or 1.1 or less. In one embodiment, Sa/Sb may be 1.0 or less, 0.9 or less, 0.8 or less, or 0.7 or less. Moreover, in another embodiment, Sa/Sb is 0.6 or less, or 0.5 or less. Specifically, if neither grooves nor through-holes nor openings are formed in the second surface fastener 40, as illustrated in FIG. 4, Sa/Sb may be 1.7 or less, 1.6 or less, 1.5 or less, or 1.1 or less. If grooves, through-holes, or openings are formed in the second surface fastener 40, Sa/Sb may be 1.0 or less, 0.9 or less, 0.8 or less, or 0.7 or less. If grooves, through-holes, or openings are formed in the second surface fastener 40 constituted by a low-profile fastener, Sa/Sb may be 0.6 or less, or 0.5 or less.

The foregoing has been a description of the second surface fasteners 40. Meanwhile, surface fasteners of any thickness may be used for the first surface fasteners 30, as described above. Alternatively, surface fasteners such as those illustrated in FIGS. 5 to 20 may be used as the first surface fasteners 30. In other words, the first surface fasteners 30 may have characteristics similar to those of the second surface fasteners 40.

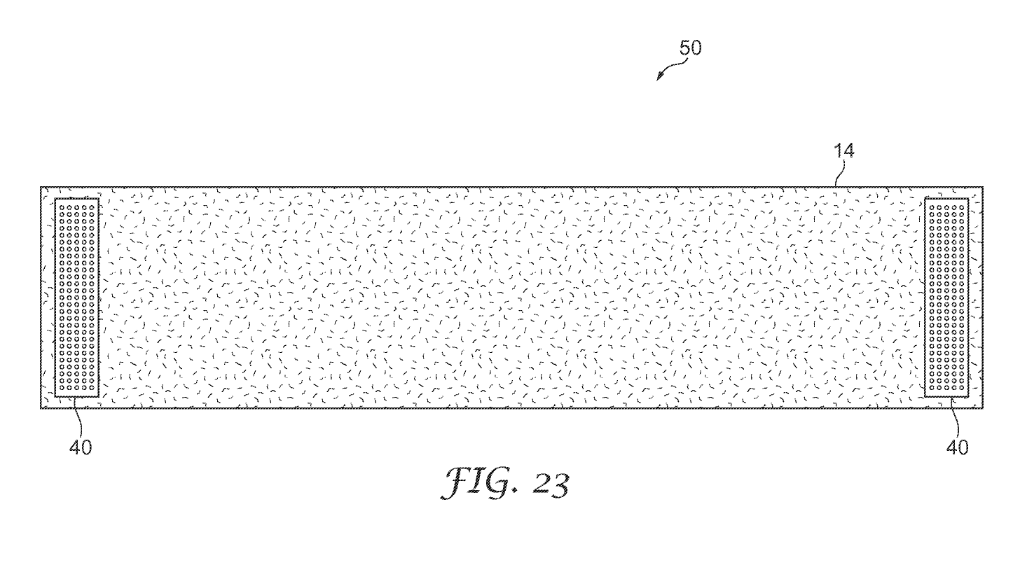

The second surface fasteners 40 may be provided singly or in the form of securing members attached to desired substrates. In the present specification, of securing members in which the second surface fasteners 40 are attached to desired substrates, those that can be individually traded are referred to as “securing element”. An example of a securing element is illustrated in FIG. 23. The securing element 50 is provided with a band-shaped loop member 14 and two second surface fasteners 40 attached to the two lengthwise ends of the loop member 14. In other words, the loop member 14 constitutes the substrate in the securing element 50. The substrate used in the securing element may be a woven fabric, nonwoven fabric, a plastic film, or a mixture thereof, as in the case of the substrate 21 described above. There is, of course, no limitation upon the structure of the second surface fasteners 40 used in the securing element; a structure such as those illustrated in FIGS. 4 to 20 may be used. Alternatively, the second surface fasteners 40 may be provided along with the first surface fasteners 30 on the securing member and may be provided as securing elements.

### EXAMPLES

The present invention will now be described in greater detail on the basis of working examples, but the present invention is not limited in any way thereto.

### Working Example 1

A strip-shaped spun-bond nonwoven fabric (manufactured by Toray Advanced Materials Korea, Inc.; length: 100 mm; width: 25 mm; basis weight: 30 g/m²) was prepared as a substrate, as was a hook member having a product name of “KJ-7891” (basis weight: 100 g/m²; base thickness: 85 μm; initial hook density: 1,600 pins/inch (note that “square inch (inch)” corresponds to 25.4 mm²); length of an oval-shaped head of a hook along a major axis direction of the oval shape (equivalent to the maximum width wb in FIG. 3): 420 μm). The substrate and hook member were then used to create a sample. Six such samples were prepared; these will be labeled 1 to 6 in the following description in order to distinguish therebetween.

The details of the respective samples were as follows. For sample 1, the prepared hook member was anchored as-is to the substrate without forming slit-shaped through-holes. For sample 2, the hook member having slit-shaped through-holes formed therein was anchored to the substrate without spreading open the through-holes. For samples 3 to 6, as illustrated in FIG. 19, the hook member was elongated by a predetermined proportion in a direction orthogonal to the slit rows (in FIG. 19, the elongation direction) to create openings, and the hook member was anchored to the substrate while maintaining the openings. The elongation rates of the hook members in samples 3 to 6 were 5%, 10%, 50%, and 80%, respectively. In other words, when anchoring the hook members to the substrates in samples 3 to 6, the lengths of the hook members were stretched 1.05 times, 1.1 times, 1.5 times, and 1.8 times, respectively. For samples 2 to 6, samples having slits extending in the lengthwise direction of the substrates and samples having slits extending in the widthwise direction thereof were prepared.

The flexibility of the samples 1 to 6 so prepared was confirmed by measuring loop stiffness using a load cell manufactured by Toyo Seiki. For this measurement, a loop was formed by bending the sample so that the hook members were positioned on the inner side of the loop formed by the sample. The circumferential length was 70 mm for all loops. The movement speed of the loops when the loops were being pressed to the load cell was 3.5 cm/min. The measurement results are shown in Table 1. In table 1, loop stiffness is the maximum measured value. The “direction parallel to the slit” corresponds to cases in which the slits extend along the circumferential direction of the loop, and the “direction orthogonal to the slit” corresponds to cases in which the slits extend in a direction orthogonal to the circumferential direction.

TABLE 1

| | Sample No. | | | | | |
|---|---|---|---|---|---|---|
| | 1 | 2 | 3 | 4 | 5 | 6 |
| Slit | No | Yes | Yes | Yes | Yes | Yes |
| Elongation rate (%) | — | 0 | 5 | 10 | 50 | 80 |
| Loop stiffness (g) | 24.5 | 18.6 | 18.6 | 16.4 | 12.0 | 10.3 |
| (direction parallel to slit) | | | | | | |
| Loop stiffness (g) | 25.7 | 8.9 | 3.9 | 2.1 | 2.4 | 1.9 |
| (direction orthogonal to slit) | | | | | | |

### Working Example 2

The flexibility of the second surface fastener described above was confirmed by measuring bending stiffness. A side panel of a commercially available diaper or a landing zone constituted by polypropylene fibers and a polypropylene film was used as the substrates of the test strips. All of the substrates had dimensions of 1 inch (25.4 mm) square. Note that landing zone refers to a portion that engages with a surface fastener (equivalent to the first surface fastener of the embodiment described above) provided on a fastening tab of the above-mentioned commercially available diaper.

Working example 2-1 was as follows. Two substrates were obtained by cutting sections from the landing zone. Moreover, a hook member having a production name of “KJ-7891” (basis weight: 100 g/m²; base thickness: 85 μm; initial hook density: 1,600 pins/inch²; length of an oval-shaped head of a hook along a major axis direction of the oval shape (equivalent to the maximum width wb in FIG. 3): 420 μm) was prepared as a second surface fastener. Slit-shaped through-holes were provided in the hook member, and the hook member was elongated by 75% in a direction orthogonal to the slit rows (in FIG. 19, the elongation direction) to create openings.

The hook member was attached to the substrate using a 25 μm-thick adhesive layer while maintaining the openings to obtain a test strip (securing member) constituted by a laminate formed from the substrate and the second surface fastener. Moreover, a piece of comparison tape (cellophane adhesive tape as specified in JIS Z 1522) of the same dimensions as the substrate was prepared, and the comparison tape was applied to another substrate to prepare a laminate for use as a test strip constituting a comparison member. Note that no slits were formed in the comparison tape.

Working example 2-2 was as follows. Two substrates were obtained by cutting sections from the side panel. Moreover, a hook member similar to that used in working example 2-1 was prepared as a second surface fastener. Slit-shaped through-holes were provided in the hook member, and the hook member was elongated by 75% in a direction orthogonal to the slit rows (in FIG. 19, the elongation direction) to create openings. The hook member was attached to the substrate using a 25 μm-thick adhesive layer while maintaining the openings to obtain a test strip (securing member) constituted by a laminate formed from the substrate and the second surface fastener. Moreover, the above-mentioned piece of comparison tape of the same dimensions as the substrate was prepared, and the comparison tape was applied to a substrate to prepare a laminate for use as a test strip constituting a comparison member. Note that no slits were formed in the comparison tape.

Working example 2-3 was as follows. Two substrates were obtained by cutting sections from the landing zone. Moreover, a hook member (basis weight: 65 g/m²; base thickness: 65 μm; initial hook density: 1,600 pins/inch²; length of an oval-shaped head of a hook along a major axis direction of the oval shape (equivalent to the maximum width wb in FIG. 3): 350 μm) was prepared as a second surface fastener. No slit-shaped through-holes were provided in the hook member. The hook member was then fused to the substrate to obtain a test strip (securing member) constituted by a laminate formed from the substrate and the second surface fastener. A test strip constituting a comparison member was obtained in the same manner as in working example 2-1.

Working example 2-4 was as follows. Two substrates were obtained by cutting sections from the side panel. Moreover, a hook member similar to that used in working example 2-3 was prepared as a second surface fastener. No slit-shaped through-holes were provided in the hook member. The hook member was then fused to the substrate to obtain a test strip (securing member) constituted by a laminate formed from the substrate and the second surface fastener. A test strip constituting a comparison member was obtained in the same manner as in working example 2-2.

Comparative example 2-1 was as follows. Two substrates were obtained by cutting sections from the landing zone. Moreover, a hook member similar to that used in working example 2-1 was prepared as a second surface fastener. However, no slit-shaped through-holes were provided in the hook member. The hook member was then fused to the substrate to obtain a test strip (securing member) constituted by a laminate formed from the substrate and the second surface fastener. A test strip constituting a comparison member was obtained in the same manner as in working example 2-1.

Comparative example 2-2 was as follows. First, two substrates were obtained by cutting sections from the side panel. Moreover, a hook member similar to that used in working example 2-1 was prepared as a second surface fastener. However, no slit-shaped through-holes were provided in the hook member. The hook member was then fused to the substrate to obtain a test strip (securing member) constituted by a laminate formed from the substrate and the second surface fastener. A test strip constituting a comparison member was obtained in the same manner as in working example 2-2.

The respective bending stiffnesses of the securing members were measured via a cantilevered bending test according to the method of TAPPIT 543 om-11 (i.e., the method illustrated in FIGS. 21, 22). Gurley Stiffness tester 4171E manufactured by Gurley Precision Instruments was used as the Gurley tester. One end of the test strip was gripped using a clamp to anchor the test strip in place, with 19.05 mm (0.75 inches) of the test strip protruding from the clamp. Moreover, the arrangement was adjusted so that 6.35 mm (0.25 inches) of the free end of the test strip came into contact with the plate-shaped member. When securing the test strips provided with second surface fasteners in working examples 2-1 and 2-2, the test strips were gripped so that the slits thereof extended in the direction from the clamps toward the rotating shaft (direction L′ in FIG. 22). In a single round of measurement, the test strip was moved back and forth in the clockwise and counterclockwise directions, and the average value of the bending stiffness in both directions was determined. Five such rounds of measurement were performed to determine average values in order to obtain the bending stiffness of each test strip. In all of working examples 2-1 to 2-4 and comparative examples 2-1 and 2-2, Sa was defined as the bending stiffness of the test strip provided with the second surface fastener (securing member), Sb was defined as the bending stiffness of the test strip provided with the comparison tape (comparison member), and the value of Sa/Sb was calculated. The measurement results are shown in Table 2.

TABLE 2

| | Sample | | | | | |
|---|---|---|---|---|---|---|
| | Working Example | | | | Comparative Example | |
| | Working | Working | Working | Working | Comparative | Comparative |
| | Example | Example | Example | Example | Example | Example |
| | 2-1 | 2-2 | 2-3 | 2-4 | 2-1 | 2-2 |
| Slit | YES | YES | NO | NO | NO | NO |
| hook member | 100 | 100 | 65 | 65 | 100 | 100 |
| basis weight | | | | | | |
| (g/m²) | | | | | | |
| Substrate | Landing | Side | Landing | Side | Landing | Side |
| | zone | panel | zone | panel | zone | panel |
| Sa/Sb | 0.69 | 0.70 | 1.44 | 1.60 | 2.40 | 2.62 |

As discussed above, a fastening system according to one aspect of the present invention is for securing a first region having a first securing member and a second region having a second securing member. The fastening system includes a first surface fastener provided on the first securing member and a second surface fastener provided on the first securing member or the second securing member. The securing member, out of the first securing member and the second securing member, having the second surface fastener provided thereon includes the second surface fastener and a substrate having the second surface fastener laminated thereon. Defining Sa as the bending stiffness of a laminated section formed by the second surface fastener and the substrate and Sb as the bending stiffness of a comparison member constituted by a laminate formed from a substrate having no second surface fastener laminated thereon and a piece of cellophane adhesive tape as specified in JIS Z 1522, the relationship Sa/Sb<2.0 holds.

In accordance with this aspect, the securing member in which the second surface fastener as described above is provided on the substrate is flexible, with the result that, when the two regions are secured, the hold yielded by the fastening system flexibly deforms in response to bending or twisting generated by movement of the member to be secured. It is thus possible to ensure reliable conformity of the securing part with respect to movement of a member to be secured even when two surface fasteners are used to increase stability against shifting. Moreover, if the fastening system is used in an absorbent article or a garment, irritation of the skin of the wearer of the absorbent article or the garment can be minimized.

In a fastening system according to another aspect, the second surface fastener may have a thickness of 60 to 380 μm. By imparting the second surface fastener with a thickness in this range (i.e., by using a low-profile fastener as the second surface fastener), a flexible securing member can be obtained when the second surface fastener is laminated onto the substrate to form the securing member. As a result, when the two region are secured, the hold yielded by the fastening system flexibly deforms in response to bending or twisting generated by movement of the member to be secured.

In a fastening system according to another aspect, grooves, slit-shaped through-holes, or openings may be formed in the second surface fastener. Forming grooves, through-holes, or openings in the second surface fastener yields a flexible securing member when the second surface fastener is laminated onto the substrate to form the securing member. As a result, when the two regions are secured, the hold yielded by the fastening system flexibly deforms in response to bending or twisting generated by movement of the member to be secured.

In a fastening system according to another aspect, the second surface fastener may be positioned at a location farther from a free end of the first securing member than the first surface fastener when the first region and the second region are secured.

An absorbent article according to an aspect of the present invention is provided with the fastening system according to any one of the aspects described above.

The foregoing has been a detailed description of the present invention with reference to the embodiments thereof. However, the present invention is not limited to the embodiments described above. Various modifications may be made to the present invention to the extent that they do not depart from the scope of the present invention.

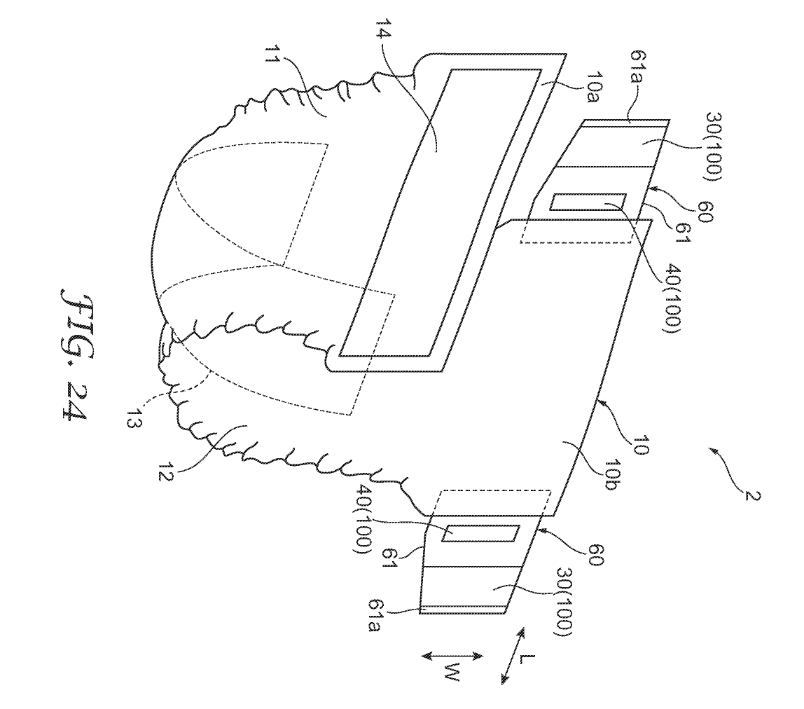

Both a first surface fastener and a second surface fastener may be provided on a single securing member. For example, both the first surface fastener and the second surface fastener may be provided on the inner surface of the dorsal side, the outer surface of the dorsal side, the inner surface of the ventral side, or the outer surface of the ventral side. The diaper (absorbent article) 2 illustrated in FIG. 24 is one such modified example. The diaper 2 includes a body section 10 like that of the embodiment described above and a pair of side panels 60 for releasably joining a ventral end 10a and a dorsal end 10b on the right and left sides of the waist of a wearer. In the diaper 2, the side panels are attached only to the dorsal end 10b. Each of the side panels 60 has a substrate 61 and a fastening system 100 (first surface fasteners 30 and second surface fasteners 40) provided on the inner surface of the substrate 61. The material used for the substrate 61 can be determined in the same way as for the substrate 21 of the embodiment described above. The distal ends of the side panels 60 function as grip sections 61a that can be easily gripped even when the first surface fasteners 30 are joined to the loop member 14. However, the first surface fasteners 30 may also be present up to the ends of the substrates 61.

Thus, in the diaper 2, the side panels 60 are the first securing members, the dorsal end 10b is the first region, and the ventral end 10a is the second securing member and the second region. Even when both the first surface fasteners 30 and the second surface fasteners 40 are provided on a single securing member, the second surface fasteners 40 are positioned at locations farther from the free ends of the securing members on which the first surface fasteners 30 are provided (in the example illustrated in FIG. 24, the side panels 60) than the first surface fasteners 30 when the two regions (in FIG. 24, the ventral end 10a and the dorsal end 10b) are secured. In this instance, the free ends of the side panels 60 are the ends with respect to the wrapping direction L on the sides opposite the dorsal end 10b.

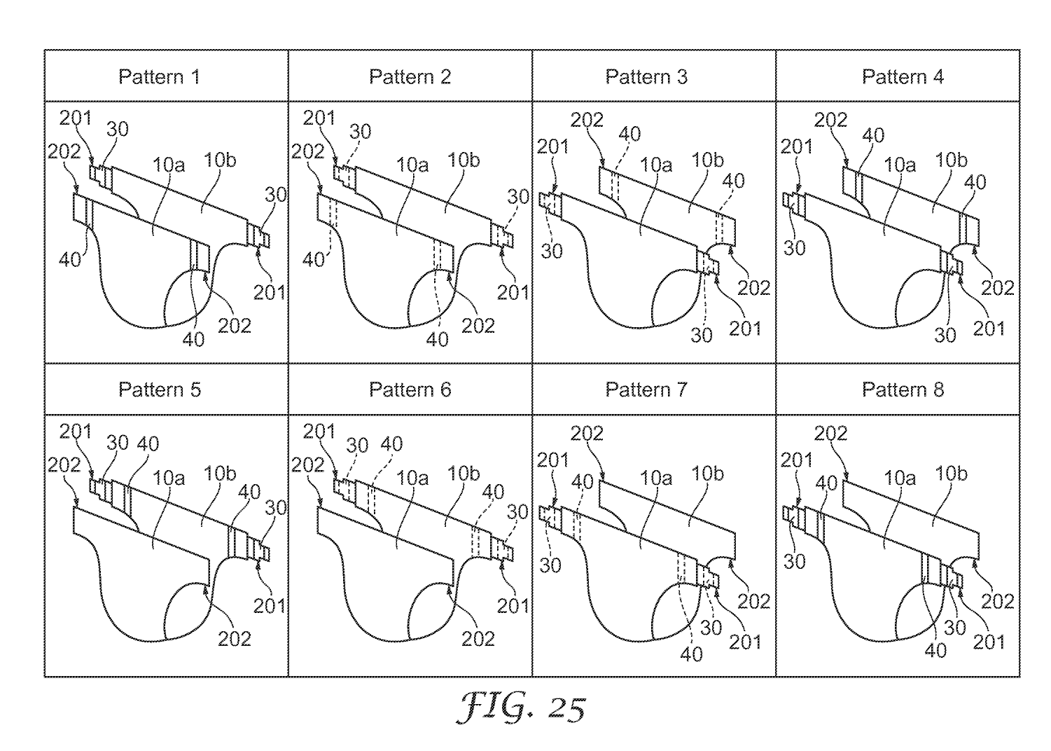

In the embodiment described above, the first surface fasteners 30 are provided on the inner surface (the surface facing the wearer) of the dorsal side and the second surface fasteners 40 are provided on the outer surface (the side opposite the inner surface) of the ventral side, but the first surface fasteners 30 and the second surface fasteners 40 are not limited to having such relative positions. A variety of arrangement patterns 1 to 8 that include embodiments such as those of the diapers 1, 2 described above are illustrated in FIG. 25. Note that, in FIG. 25, the ventral end 10a and the dorsal end 10b are indicated in order to make the orientation of the diapers more apparent.

In patterns 1 to 4, the first surface fasteners 30 are present in first regions 201, and the second surface fasteners 40 are present in second regions 202. The respective surface fasteners are disposed as follows.

In pattern 1, the first surface fasteners 30 are positioned on the inner surface of the dorsal side, and the second surface fasteners 40 are positioned on the outer surface of the ventral side.

In pattern 2, the first surface fasteners 30 are positioned on the outer surface of the dorsal side, and the second surface fasteners 40 are positioned on the inner surface of the ventral side.

In pattern 3, the first surface fasteners 30 are positioned on the inner surface of the ventral side, and the second surface fasteners 40 are positioned on the outer surface of the dorsal side.

In pattern 4, the first surface fasteners 30 are positioned on the outer surface of the ventral side, and the second surface fasteners 40 are positioned on the inner surface of the dorsal side.

In patterns 5 to 8, the first surface fasteners 30 and the second surface fasteners 40 are present in the first regions 201. The respective surface fasteners are disposed as follows.

In pattern 5, the first surface fasteners 30 and the second surface fasteners 40 are positioned on the inner surface of the dorsal side.

In pattern 6, the first surface fasteners 30 and the second surface fasteners 40 are positioned on the outer surface of the dorsal side. In pattern 7, the first surface fasteners 30 and the second surface fasteners 40 are positioned on the inner surface of the ventral side.

In pattern 8, the first surface fasteners 30 and the second surface fasteners 40 are positioned on the outer surface of the ventral side.

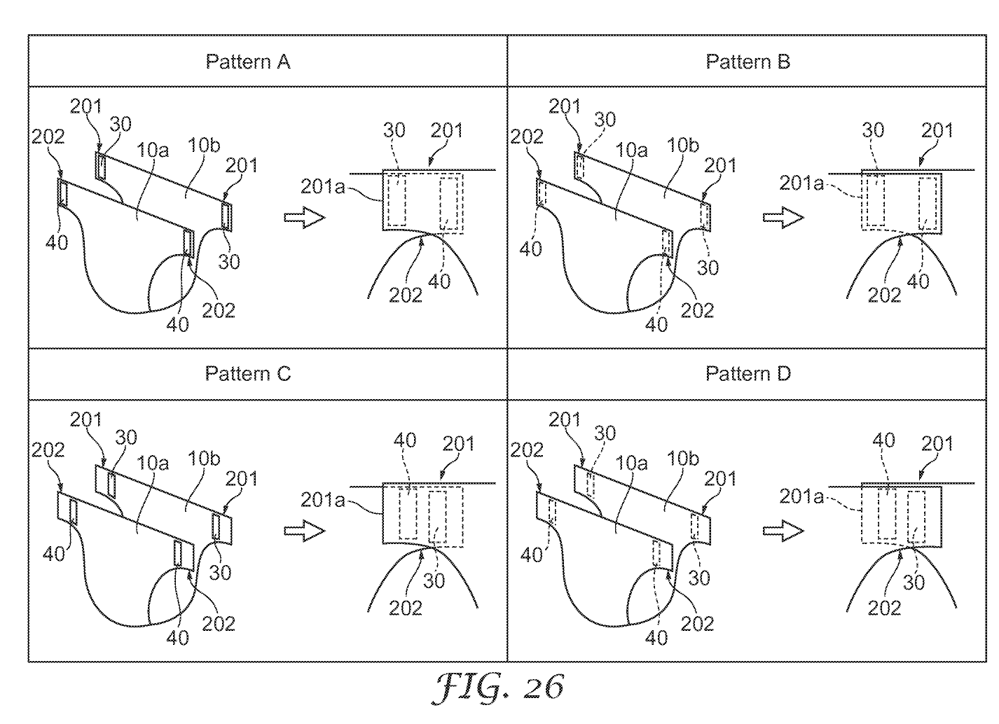

In the present fastening system, it is not essential that the second surface fastener be positioned at a location farther from the free end of the first securing member than the first surface fastener when the first region and the second region are secured. A variety of arrangement patterns A to D that include embodiments such as those of the diapers 1, 2 described above are illustrated in FIG. 26. Note that, in FIG. 26 as well, the ventral end 10a and the dorsal end 10b are indicated in order to make the orientation of the diapers more apparent.

In all of patterns A to D, the first regions 201 having the first surface fasteners 30 are positioned on the dorsal side, and the second regions 202 having the second surface fasteners 40 are provided on the ventral side. Moreover, the first regions 201 and the second regions 202 are side panels.

In pattern A, the respective surface fasteners are positioned on free ends of the side panels with respect to the wrapping direction. The first surface fasteners 30 are positioned on the inner surface of the dorsal side, and the second surface fasteners 40 are positioned on the outer surface of the ventral side. In pattern A, when the first regions 201 and the second regions 202 are overlapped, the second surface fasteners 40 are positioned at locations farther from the free ends 201a of the first regions (first securing members) 201 than the first surface fasteners 30.

In pattern B, the respective surface fasteners are positioned on free ends of the side panels with respect to the wrapping direction. The first surface fasteners 30 are positioned on the outer surface of the dorsal side, and the second surface fasteners 40 are positioned on the inner surface of the ventral side. In pattern B, when the first regions 201 and the second regions 202 are overlapped, the second surface fasteners 40 are positioned at locations farther from the free ends 201a of the first regions (first securing members) 201 than the first surface fasteners 30. In pattern C, the respective surface fasteners are positioned at the bases of the side panels with respect to the wrapping direction. The first surface fasteners 30 are positioned on the inner surface of the dorsal side, and the second surface fasteners 40 are positioned on the outer surface of the ventral side. In pattern C, when the first regions 201 and the second regions 202 are overlapped, the first surface fasteners 30 are positioned at locations farther from the free ends 201a of the first regions (first securing members) 201 than the second surface fasteners 40.

In pattern D, the respective surface fasteners are positioned at the bases of the side panels with respect to the wrapping direction. The first surface fasteners 30 are positioned on the outer surface of the dorsal side, and the second surface fasteners 40 are positioned on the inner surface of the ventral side. In pattern D, when the first regions 201 and the second regions 202 are overlapped, the first surface fasteners 30 are positioned at locations farther from the free ends 201a of the first regions (first securing members) 201 than the second surface fasteners 40.

As described above, at least one of the first surface fastener and the second surface fastener can be a loop member. In this case, the height of the loop bodies (engaging part) formed on the base can be established to be the same as the hooks in the above embodiment. If at least one of the first surface fastener and the second surface fastener is a loop member, a hook member is provided at a section engaging with the loop bodies thereof.

In the embodiments described above, the fastening system is applied to a diaper as one example of an absorbent article, but the fastening system may also be applied to other articles. For example, the fastening system may also be applied to a sanitary article/hygienic article, such as a sanitary napkin. The fastening system may also be applied to a bandage or the like. Note that the fastening system itself can also be used as a bandage. Moreover, the fastening system can also be applied to uses such as a bundling band used to bundle and organize a plurality of cables, a temporary securing member for securing a plurality of articles together, or the like.

### REFERENCE NUMERALS

1, 2 Absorbent article (diaper)
10 Body section
10a Ventral end
10b Dorsal end
14 Loop member
16 Ventral side panel
17 Dorsal side panel
21 Substrate
30 First surface fastener
40 Second surface fastener
41 Base
42 Hook
43 Groove or through-hole
43a Slit-shaped through-hole
43b Slit-shaped groove
44 Opening
45 Strand
46 Bridge section
50 Securing element
60 Side panel
100 Fastening system

1. A fastening system for securing a first region having a first securing member and a second region having a second securing member, the fastening system comprising:
a first surface fastener provided on the first securing member; and
a second surface fastener provided on the first securing member or the second securing member;
that securing member, out of the first securing member and the second securing member, having the second surface fastener provided thereon including the second surface fastener and a substrate having the second surface fastener laminated thereon; and
defining Sa as bending stiffness of a laminated section formed by the second surface fastener and the substrate and Sb as bending stiffness of a comparison member constituted by a laminate formed from the substrate having no second surface fastener laminated thereon and a piece of cellophane adhesive tape as specified JIS Z 1522, a relationship Sa/Sb<2.0 holds.

2. The fastening system according to claim 1, wherein
the second surface fastener has a thickness of from 60 to 380 μm.

3. The fastening system according to claim 1, wherein
a groove, slit-shaped through-hole, or opening is formed in the second surface fastener.

4. The fastening system according to claim 1, wherein
the second surface fastener is positioned at a location farther from a free end of the first securing member than the first surface fastener when the first region and the second region are secured.

5. An absorbent article comprising the fastening system described in claim 1.
